(19) United States
(12) Patent Application Publication
(10) Pub. No.: US 2005/0106160 A1
(43) Pub. Date: May 19, 2005

(54) Hiv-1 envelope glycoproteins stabilized by flexible linkers as potent entry inhibitors and immunogens

(76) Inventors: Dimiter Stanchev Dimitrov, Rockville, MD (US); Yen-Hung Chow, Fairplay, MD (US); Sanjay Kumar Phogat, Frederick, MD (US); Christopher Charles Broder, Rockville, MD (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/506,651

(22) PCT Filed: Mar. 5, 2002
(86) PCT No.: PCT/US02/07144

(51) Int. Cl. A61K 39/21; C07K 14/16

(52) U.S. Cl. 424/188.1; 530/395

(57) ABSTRACT

This invention relates generally to immune responses to human immunodeficiency virus coat protein gp160 presented in the form of antigenic compositions, nucleic acids encoding human immunodeficiency virus coat proteins, and vaccines. The invention also relates to methods for production of antigenic compositions containing human immunodeficiency virus coat protein, nucleic acids encoding human immunodeficiency virus coat proteins, and human immunodeficiency virus vaccines. The invention comprises gp120 and gp41 subunits of the human immunodeficiency virus coat protein covalently linked through a peptide linker, as well as additional complexes including those comprising the human immunodeficiency virus coat protein and it's natural cellular receptor molecules.

### CROSS-REFERENCES TO RELATED APPLICATIONS

NOT APPLICABLE

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### FIELD OF THE INVENTION

This invention relates generally to immune responses and more particularly to immune responses to human immunodeficiency virus coat proteins presented in the form of antigenic compositions, nucleic acids encoding human immunodeficiency virus coat protein gp160, and vaccines. The invention also relates to methods for production of antigenic compositions containing human immunodeficiency virus coat proteins, nucleic acids encoding human immunodeficiency virus coat protein gp160, and human immunodeficiency virus vaccines.

### BACKGROUND OF THE INVENTION

The human immunodeficiency virus (HIV) is the primary cause of the slowly degenerative immune system disease termed acquired immune deficiency syndrome (AIDS) (Barre-Sinoussi, F. et al., Science 220:868-870 (1983); Gallo, R et al., Science 224:500-503 (1984)). There are at least two distinct types of HIV: HIV-1 (Barre-Sinoussi. et al., Science 220:868-870 (1983); Gallo et al., Science 224:500-503 (1984)) and HIV-2 (Clavel et al., Science 233:343-346 (1986); Guyader et al., Nature 326:662-669 (1987)). Further, a large amount of genetic heterogeneity exists within populations of each of these types. Infection of human CD-4.sup.⁺ T-lymphocytes with an HIV virus leads to depletion of the cell type and eventually to opportunistic infections, neurological dysfunctions, neoplastic growth, and ultimately death.

The HIV viral particle consists of a viral core, composed of capsid proteins, that contains the viral RNA genome and those enzymes required for early replicative events. Myristylated Gag protein forms an outer viral shell around the viral core, which is, in turn, surrounded by a lipid membrane envelope derived from the infected cell membrane. The HIV envelope surface glycoproteins are synthesized as a single 160 kDa precursor protein that is cleaved by a cellular protease during viral budding into two glycoproteins, gp41 and gp120. gp41 is a transmembrane protein and gp120 is an extracellular protein which remains non-covalently associated with gp41, possibly in a triaminic or multimeric form (Hammarskjold, M. and Rekosh, D., Biochem. Biophys. Acta 989:269-280 (1989)).

HIV is targeted to CD4.sup.⁺ cells because the CD-4 cell surface protein acts as the cellular receptor for the HIV-1 virus (Dalgleish et al., Nature 312:763-767 (1984); Klatzmann et al., Nature 312:767-768 (1984); Maddon et al., Cell 47:333-348 (1986)). Viral entry into cells is dependent upon gp120 binding the cellular CD-4.sup.⁺ receptor molecules (McDougal et al., Science 231:382-385 (1986); Maddon et al., Cell 47:333-348 (1986)) and thus explains HIV's tropism for CD4.sup.⁺ cells, while gp41 anchors the envelope glycoprotein complex in the viral membrane.

HIV infection is pandemic and HIV associated diseases represent a major world health problem. Considerable attention is being given to the development of vaccines for the treatment of HIV infection. This attention has been largely directed towards the HIV-1 envelope proteins (gp160, gp120, gp41) which have been shown to be the major antigens for anti-HIV antibodies present in AIDS patients (Barin et al., Science 228:1094-1096 (1985)). To this end, several groups have begun to use various portions of gp160, gp120, and/or gp41 as immunogenic targets for the host immune system. See for example, Ivanoff, L. et al., U.S. Pat. No. 5,141,867; Saith, G. et al., WO 92/22,654; Shafferman, A., WO 91/09,872; Formoso, C. et al., WO 90/07,119. To date, none of these approaches has resulted in an effective preventative preparation. Thus, although a great deal of effort is being directed to the design and testing of vaccine preparations, a truly effective, non-toxic treatment has yet to be produced.

### BRIEF SUMMARY OF THE INVENTION

The invention provides a human immunodeficiency virus antigenic composition comprising a human immunodeficiency virus envelope glycoprotein 160 having a gp120 subunit and a gp41 subunit where the carboxyl-terminal end of gp120 is covalently linked through a peptide linker of at least 5 amino acids, to the amino-terminal end of gp41. The human immunodeficiency virus envelope glycoprotein 160 may also be a truncated form, the truncation being at a position within 5 amino acids either side of amino acid 683 in SEQ ID NO:2. This truncated form comprises gp120 and the extracellular subunits of gp41.

A preferred aspect of the antigenic composition is that the peptide linker is between 6 and 29 and more preferably between 15 and 26 amino acids in length. The peptide linker may also be comprised of repeating units such as those disclosed in SEQ ID NOS:12, 13 and 14, more preferably the sequence set out in SEQ ID NO:10 or, most preferably the sequence set out in SEQ ID NO:11.

Another preferred aspect of the human immunodeficiency virus envelope glycoprotein 160 is that it has at least 70% amino acid sequence identity to sequence SEQ ID NO:2, more preferably being identical to SEQ ID NO:2. Where the truncated form of the human immunodeficiency virus envelope glycoprotein 160 is used, it is preferable that the truncated sequence be at least 70% identical to the amino acid sequence of SEQ ID NO:4, more preferably, identical SEQ ID NO:4.

Another aspect of the invention provides that the gp120 subunit and the gp41 subunit can be from the same or different human immunodeficiency virus strains.

The invention also provides a method of manufacturing a human immunodeficiency virus antigenic composition comprising a human immunodeficiency virus envelope glycoprotein 160 having a gp120 subunit and a gp41 subunit where the carboxyl-terminal end of gp120 is covalently linked through a peptide linker of at least 5 amino acids, to the amino-terminal end of gp41. The human immunodeficiency virus envelope glycoprotein 160 may also be a truncated form, the truncation being at a position within 5 amino acids either side of amino acid 683 in SEQ ID NO:2. The method includes the steps of obtaining nucleic acids that encode gp120 and gp41. A peptide linker is next introduced in frame between the gp120 and the gp41 coding segments. This peptide linker is between 6 and 29 amino acids. The resulting nucleic acid is next operably linked to regulatory sequences of an appropriate expression cassette. The expression cassette is then introduced into a mammalian host cell and the host cell cultured in a manner that promotes expression of the human immunodeficiency virus antigenic composition. Finally, the method provides means for isolating the antigenic composition from the host cell. The preferred embodiments of the antigenic composition produced by this method are the same as those noted above for the antigenic composition itself.

The invention also provides a vaccine for protecting a human from human immunodeficiency virus infection. This vaccine comprises an aliquot amount of the human immunodeficiency virus antigenic composition described above, presented in a suitable, sterile, pharmaceutically acceptable carrier. Preferably, the aliquot amount of human immunodeficiency virus antigenic composition present in the vaccine is between 0.5 and 1 milligrams per milliliter of sterile pharmaceutically acceptable carrier. Alternatively, the aliquot amount of human immunodeficiency virus antigenic composition can be in a lyophilized state. An additional preferable embodiment includes formulating the vaccine with one or more glycoprotein 160 ligands chosen from the group consisting of CD4, CCR5 and CXCR4, which are capable of forming a complex with the antigenic coat protein encoded by gp160.

The invention further provides a method of protecting a human from human immunodeficiency virus infection. This method comprises administering the human immunodeficiency virus antigenic composition described above in an amount sufficient to be effective in immunizing the individual against infection by the virus, or capable of neutralizing human immunodeficiency virus coming into contact with the antigenic composition. The antigenic composition can optionally be formulated into a creme, lotion, douche or into the lining of a condom. The effective amount administered is preferably between 1 μg/kg and 20 μg/kg per dose per inoculation. A preferred embodiment of the method is the inclusion one or more glycoprotein 160 ligands, such as CD4, CCR5 or CXCR4. When these ligands are included, they preferably are present in a molar ration of between 3:1 and 1:3 relative to the previously described antigenic.

A nucleic acid comprising a coding sequence for a human immunodeficiency virus envelope glycoprotein 160 having a gp120 subunit and a gp41 subunit where the carboxyl-terminal end of gp120 is covalently linked through a peptide linker of at least 5 amino acids to the amino-terminal end of gp41 is also provided by the invention. The preferences for the proteins encoded by this nucleic acid are identical to those detailed for the antigenic composition noted above. To facilitate expression in Eukaryotic cells, it is preferable that the nucleic acid is operably linked to regulatory sequences for expression of DNA in eukaryotic.

Embodiments of the nucleic acid include both truncated and untruncated versions of the gp160 protein. As described above for the antigenic composition, truncated forms of gp160 comprise a gp41 extracellular subunit where the transmembrane subunit has been lost Most preferably, truncated forms of gp160 have the sequence listed in SEQ ID NO:3, while untruncated forms are of the sequence listed in NO:1.

In addition to coding regions for a gp160 and regulatory sequences, embodiments of the nucleic acid also include coding sequence and necessary regulatory sequences for the expression of one or more glycoprotein 160 ligands chosen from the group consisting of CD4, CCR5 and CXCR4.

Preferred embodiments of the nucleic acid comprise a peptide linker of between 6 and 29 amino acids, most preferably between 15 and 26 amino acids in length. Still more preferably, the peptide linker may be comprised of repeating units such as those set out in SEQ ID NOS:12, 13, and 14, or simply one of the sequences set out in SEQ ID NOS:10 and 11.

Another aspect provided by the present invention is a live recombinant vaccine comprising an nucleic acid comprising a coding sequence for a human immunodeficiency virus envelope glycoprotein 160 having a gp120 subunit and a gp41 subunit where the carboxyl-terminal end of gp120 is covalently linked through a peptide linker of at least 5 amino acids to the amino-terminal end of gp41. The preferences for this coding sequence of the live virus are identical to those described for the nucleic acid above. The live recombinant vaccine can be formulated with one or more glycoprotein 160 ligands chosen from the group consisting of CD4, CCR5 and CXCR Such formulations allow for the formation of complexes between the viral gp160 coat protein and the ligands of the group. When included, the gp160 ligands are present in the formulation in a molar ratio of between 3:1 and 1:3 for each ligand species of the composition.

### BRIEF DESCRIPTION OF THE DRAWINGS

FIG. 1 illustrates the competitive effect of gp120-gp41 fusion proteins constructed with peptide linkers of indicated lengths on cell fusion. The level of luciferase activity is correlative to the percentage of cells successfully fusing in the assay.

FIG. 2 shows the cleavage sites in the gp160 (env 89.6) protein which delimit both the truncated and untruncated forms of gp41.

### DETAILED DESCRIPTION

I. Introduction

This invention provides human immunodeficiency (HIV)-1 envelope glycoprotein (Env, gp120-gp41) molecule which is stabilized by the insertion of a variable length polypeptide linker between its component gp120 and gp41, forming a fusion protein, gp120-gp41. By tethering the carboxy-terminal end of gp120 to the amino-terminal end of gp41 with a flexible polypeptide linker, the present invention (i) stabilizes the interaction between gp120 and gp41, and (ii) enhances and stabilizes the exposure of conserved antibody epitopes. These aspects of the invention increase the usefulness of gp120-gp41 in both research and clinical applications by enhancing the antigenicity of both the isolated molecule and the complexes formed between gp120-gp41 and the CD4 and HIV-1 coreceptors.

Soluble variants of the envelope protein complex provided by the invention are constructed of gp120 tethered to a truncated version of gp41. This truncated version of gp41 comprises the extraceUular subunit of the native protein, or it's equivalent.

The invention also provides methods and compositions for preventing and treating aids infection. Compositions include vaccines, both protein-based and DNA-based, for immunizing serio-negative individuals. These vaccines can also be used to delay or halt the progress of an existing infection. Other compositions include creams, ointments, sauves and other topical preparations to neutralize fluids comprising the HIV virus. Compositions for suppositories and pills are also provided. These compositions can be enhanced by addition of molecules specifically recognized by the gp160 viral coat protein. When included, the molecules specifically recognized by the gp160 glycoprotein are present in the formulation in a molar ratio of between 3:1 and 1:3 for each ligand species of the composition, relative to the gp120-gp41 fusion protein.

The peptide linker of the invention can be any length greater than 5 amino acids. By way of example, a preferable length is between 6 and 29 amino acids, more preferably between 15 and 26 amino acids in length. Any peptide may, however, be used as a linker in the invention, provided that the resulting gp120-gp41 fusion protein is capable of inhibiting syncytia formation in the assay of Example 2.

Definitions

As used herein, the following terms have the meanings ascribed to them unless specified otherwise.

The term “gp160” refers to the human immunodeficiency virus-1 (HIV) gene encoding the HIV envelope glycoprotein illustrated by example in SEQ ID:1. gp160 comprises two coding regions, one encoding the 120 kDa (gp120) of the envelope glycoprotein and the other encoding the 41 kDa subunit (gp41) which includes a transmembrane region and a cytoplasmic tail. In the context of this invention, the term “gp160” also refers to a truncated version of gp160 alternatively termed “gp140”. This truncated version lacks the transmembrane subunit and the cytoplasmic tail which is defined as the 3′ end of the gp160 gene sequence, beginning within 5 amino acids either side of residue 684 as noted in SEQ ID NO:3.

“gp120” or “gp120 subunit” refers to a sequence, including variants, mutants, and orthologs, both isolated and within a larger protein (e.g., gp160) or protein complex (e.g., the mature human immunodeficiency virus-1 (HIV) envelope glycoprotein) which is about 120 kDa and characterized by: (1) having an amino acid subsequence that has greater than about 60% amino acid sequence identity, 65%, 70%, 75%, 80%, 85%, 90%, preferably 95%, 96%, 97%, 98%, 99% or greater amino acid sequence identity, to the sequence of the gp120 region of SEQ ID NO:2 or 4; (2) binding to antibodies, e.g., polyclonal antibodies, raised against an immunogen comprising an amino acid sequence of SEQ ID NO:2 or 4, and conservatively modified variants thereof; (3) specifically hybridizing under stringent hybridization conditions to a sequence of SEQ ID NOS:1 or 3 and conservatively modified variants thereof; and (4) having a nucleic acid subsequence that has greater than about 85%, preferably greater than about 90%, 95%, 98%, 99%, or higher nucleotide sequence identity to the gp120 regions of SEQ ID NO:1 or 3. For purposes of this invention, the terms “regions”, “subunits” and “subunits” are used interchangeably. In the context of the primary sequence of gp160 (SEQ ID NO: 2) or gp140 (SEQ ID NO:4), or variants therefrom as defined herein, the gp120 region is that portion of the protein delimited by the first 508 amino acids of either gp160 or gp140, plus or minus 5 amino acids added to, or deleted from, the ends of this sequence.

“gp41” or “g41 subunit” refers to a sequence, including variants, mutants, and orthologs, both isolated and within a larger protein (e.g., gp160) or protein complex (e.g., the mature human immunodeficiency virus-1 (HIV) envelope glycoprotein) which is at least 41 kDa and characterized by: (1) having an amino acid subsequence that has greater than about 60% amino acid sequence identity, 65%, 70%, 75%, 80%, 85%, 90%, preferably 95%, 96%, 97%, 98%, 99% or greater amino acid sequence identity, to the sequence of the gp41 region of SEQ ID NO:2 or 4; (2) binding to antibodies, e.g., polyclonal antibodies, raised against an immunogen comprising an amino acid sequence of SEQ ID NO:2 or 4, and conservatively modified variants thereof; (3) specifically hybridizing under stringent hybridization conditions to a sequence of SEQ ID NOS:1 and 3 and conservatively modified variants thereof, (4) having a nucleic acid subsequence that has greater than about 85%, preferably greater than about 90%, 95%, 98%, 99%, or higher nucleotide sequence identity to the gp41 sequence subunits of SEQ ID NO:1 or SEQ ID NO:3. In the context of the primary sequence of gp160 (SEQ ID NO: 2), or variants therefrom as defined herein, the gp41 region is that portion of the protein delimited by amino acid residue 509 and the carboxy terminus of the gp160 primary sequence plus or minus 5 amino acids added to, or deleted from, the ends of this sequence. Alternatively, the gp41 subunit is defined as that portion of either the gp160 (SEQ ID NO:2) or gp140 (SEQ ID NO:4) protein, or variants therefrom as defined herein, originating at the furin (or related subtilisin-like endoprotease) cleavage site (between residues 508-509) and extending to the carboxyl end of the protein.

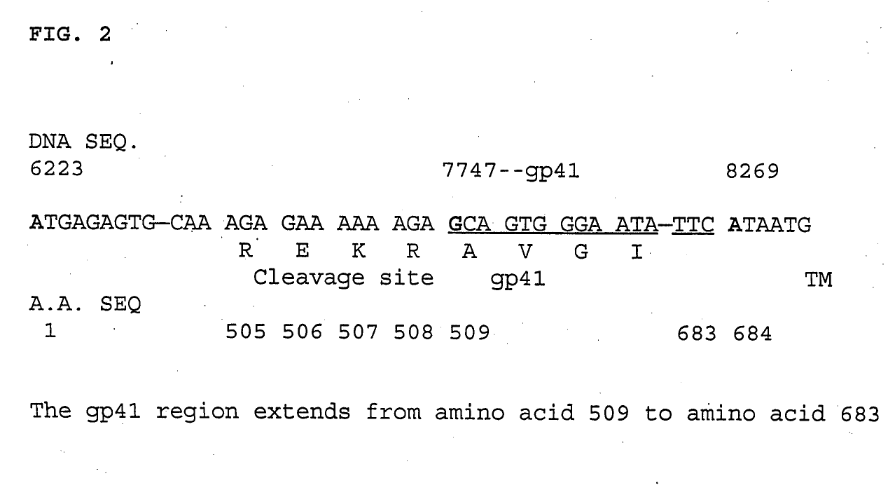

The “extracellular subunit” of gp41 is defined as the 3′ end of the gp160 gene sequence, beginning within 5 amino acids either side of residue 509 and ending 5 amino acids either side of residue 684, as noted in FIG. 2, or variants therefrom as defined herein.

“peptide linker” refers to any heterologous polypeptide of at least 6 amino acids in length, which when inserted between the carboxy-terminal end of gp120 and the amino-terminal end of gp41 yields a functional protein capable of inhibiting syncytia formation in the assay of Example 2. The peptide linker is preferably inserted within 5 amino acid residues either side of residue 509 in gp140 (FIG. 2 and SEQ ID NOS:7 and 8), although other insertion positions are possible. The term “peptide linker nucleic acid” refers to a nucleic acid encoding the peptide linker.

“regulatory sequences” refers to those sequences, both 5′ and 3′ to a structural gene, that are required for the transcription and translation of the structural gene in the target host organism. Regulatory sequences include a promoter, ribosome binding site, optional inducible elements and sequence elements required for efficient 3′ processing, including polyadenylation. When the structural gene has been isolated from genomic DNA, the regulatory sequences also include those intronic sequences required for splicing of the introns as part of mRNA formation in the target host.

“Extracellular subunit” refers to those parts of a cellular structure located outside of a cell. The “extracellular subunit” can also include short (up to 5) amino acids stretches which physically interact with the cell membrane.

The terms “fusion proteins”, “proteins of the invention”, “HIV envelope fusion proteins”, and “HIV envelope fusion glycoproteins” are synonymous in the context of this invention, and refer to proteins which inhibit syncytia formation in the assay of example 2. These terms refer structurally to proteins that comprise the carboxy-terminal end of gp120 being covalently linked through a peptide linker of at least 6 amino acids, to the amino-terminal end of gp41.

“Ligand-receptor complexes” or simply “complexes” refers to a specific association between a fusion protein and the extracellular subunit of HIV receptors.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2′-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. BiOL Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A particular nucleic acid sequence also implicitly encompasses “variant sequences.” Similarly, a particular protein encoded by a nucleic acid implicitly encompasses any protein encoded by a strain variant of that nucleic acid. “Variant sequences,” as the name suggests, are gene variations within a gene family. Such differences are most striking for viral strains isolated from different continents and presumable arise from different selection pressures in different locals and different hosts. All variant genes show at least 70% nucleic acid identity within the gene family.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are preferably directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises (e.g., a fusion protein).

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment Typically, the expression vector includes a nucleic acid to be transcribed operably linked to a promoter.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, 65%, 70%, 75%, 80%, preferably 85%, 90%, 91%, 92%, 93%, 94%, 95%, 96%, 97%, 98%, 99% or higher identity to an amino acid sequence such as SEQ ID NO:2 or a nucleotide sequence such as SEQ ID NO:1 or SEQ ID NO:3), when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Preferably, the identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of HIV envelope glycoproteins, fusion proteins comprising envelope glycoproteins and nucleic acid sequences encoding the same, the BLAST and BLAST 2.0 algorithms and the default parameters discussed below are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman, Adv. Appl. Math. 2:482 (1981), by the homology alignment algorithm of Needleman and Wunsch, J. Mol. Biol. 48:443 (1970), by the search for similarity method of Pearson and Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nln.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915 (1989)) alignments (B) of 50, expectation (E) of 10, M=5, N4, and a comparison of both strands.

The BLAST algorithm also performs a statistical analysis of the similarity between two sequences (see, e.g., Karlin and Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993). Generally, stringent conditions are selected to be about 5-10° C. lower than the thermal melting point (Tₘ for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For high stringency hybridization, a positive signal is at least two times background, preferably 10 times background hybridization. Exemplary high stringency or stringent hybridization conditions include: 50% formamide, 5×SSC and 1% SDS incubated at 42° C. or 5×SSC and 1% SDS incubated at 65° C., with a wash in 0.2×SSC and 0.1% SDS at 65° C.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993)). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of monoclonal or polyclonal antibodies, any technique known in the art can be used (see, e.g., Kohler and Milstein, Nature 256:495497 (1975); Kozbor et al., Immunology Today 4:72 (1983); Cole et al., pp. 77-96 in Monoclonal Antibodies and Cancer Therapy, Alan R. Liss, Inc. (1985)). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

An “anti-fusion protein” antibody is an antibody or antibody fragment that specifically binds a polypeptide encoded by a recombinant HIV envelope fusion protein gene, cDNA, or a subsequence thereof.

The term “immunoassay” is an assay that uses an antibody to specifically bind an antigen. The immunoassay is characterized by the use of specific binding properties of a particular antibody to isolate, target, and/or quantify the antigen.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified substantially bind in a significant amount to other proteins present in the sample. Specific binding to an antibody under such conditions may require an antibody that is selected for its specificity for a particular protein. For example, polyclonal antibodies raised to an envelope glycoprotein, as shown in SEQ ID NO:2, or variants, or portions thereof, can be selected to obtain only those polyclonal antibodies that are specifically immunoreactive with the envelope glycoprotein and not with other proteins. This selection may be achieved by subtracting out antibodies that cross-react with other molecules. In addition, polyclonal antibodies raised to envelope glycoprotein strain variants, orthologs, and conservatively modified variants can be selected to obtain only those antibodies that recognize the envelope glycoprotein, but not other proteins. A variety of immunoassay formats may be used to select antibodies specifically immunoreactive with a particular protein. For example, solid-phase ELISA immunoassays are routinely used to select antibodies specifically immunoreactive with a protein (see, e.g., Harlow and Lane, Antibodies, A Laboratory Manual (1988) for a description of immunoassay formats and conditions that can be used to determine specific immunoreactivity). Typically a specific or selective reaction will be at least twice background signal or noise and more typically more than 10 to 100 times background.

The phrase “selectively associates with” refers to the ability of a nucleic acid to “selectively hybridize” with another as defined above, or the ability of an antibody to “selectively (or specifically) bind to a protein, as defined above.

By “host cell” is meant a cell that contains an expression vector and supports the replication or expression of the expression vector. Host cells are mammalian cells such as CHO, HeLa and the like, e.g., cultured cells, explants, and cells in vivo.

Isolating Genes Encoding HIV Envelope Glycoproteins

General Recombinant DNA Methods

The nucleic acid sequences encoding HIV envelope glycoproteins may be obtained by recombinant DNA methods, such as screening reverse transcripts of mRNA, or screening genomic libraries from any HIV-infected cell or HIV isolate. The DNA may also be obtained by synthesizing the DNA from published sequences using commonly available techniques such as solid phase phosphoramidite triester method first described by Beaucage and Caruthers, Tetrahedron Letts. 22:1859-1862 (1981), using an automated synthesizer, as described in Van Devanter et al., Nucleic Acids Res. 12:6159-6168 (1984). Synthesis may be advantageous because unique restriction sites may be introduced at the time of preparing the DNA, thereby facilitating the use of the gene in vectors containing restriction sites not otherwise present in the native source. Furthermore, any desired site modification in the DNA may be introduced by synthesis, without the need to further modify the DNA by mutagenesis.

Purification of oligonucleotides is by either native acrylamide gel electrophoresis, agarose electrophoresis or by anion-exchange HPLC as described in Pearson and Reanier, J. Chrom. 255:137-149 (1983), depending upon the size of the oligonucleotide and other characteristics of the preparation. The sequence of cloned genes and synthetic oligonucleotides can be verified using, e.g., the chain termination method for sequencing double-stranded templates as described by Wallace et al., Gene 16:21-26 (1981).

Processes for producing recombinant proteins for purification by the methods of the present invention will employ, unless otherwise indicated, conventional molecular biology, microbiology, and recombinant DNA techniques within the skill of the art. Such techniques are explained fully in the literature. See e.g., Maniatis, Fritsch and Sambrook, Molecular Cloning: A Laboratory Manual, 2nd Ed. (1989); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. 1985); Transciption And Translation (B. D. Hames and S. J. Higgins eds. 1984); Animal Cell Culture (R. I. Freshney ed. 1986); Immobilized Cells And Enzymes (IRL Press, 1986); B. Perbal, A Practical Guide To Molecular Cloning (1984); Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)).

Cloning Methods for the Isolation of Nucleotide Sequences Encoding HIV Envelope Glycoproteins

In general, DNA encoding the envelope glycoproteins described herein can be obtained by constructing a cDNA library from mRNA recovered from field or laboratory isolates and (1) screening with labeled DNA probes encoding portions of the envelope glycoprotein sought in order to detect clones in the cDNA library that contain homologous sequences or (2) amplifying the cDNA using polymerase chain-reaction (PCR) and subcloning and screening with labeled DNA probes. Clones can then be analyzed by restriction enzyme analysis, agarose gel electrophoresis sizing and nucleic acid sequencing so as to identify full-length clones and, if full-length clones are not present in the library, recovering appropriate fragments from the various clones and ligating them at restriction sites common to the clones to assemble a clone encoding a full-length molecule. DNA probes for envelope glycoproteins are common in the art and can be prepared from the genetic material set forth in SEQ ID NOS:1 and 3. Any sequences missing from the 5′ end of the cDNA may be obtained by the 3′ extension of the synthetic oligonucleotides complementary to sequences encoding the protein using mRNA as a template (so-called primer extension), or homologous sequences may be supplied from known cDNAs. Polynucleic acid sizes are given in either kilobases (Kb) or base pairs (bp). These sizes are estimates derived from agarose or acrylamide gel electrophoresis, from sequenced nucleic acids, or from published DNA sequences.

Amplification techniques using primers can also be used to isolate HIV envelope glycoproteins from DNA or RNA. Suitable primers are commonly available in the art, or can be derived from SEQ ID NOS:1 or 3, then synthesized by conventional solid-phase techniques common in the art and described. Primers can be used, e.g., to amplify either the full length sequence or a probe of one to several hundred nucleotides, which is then used to screen a library for full-length HIV envelope glycoproteins.

Nucleic acids encoding HIV envelope glycoproteins can also be isolated from expression libraries using antibodies as probes. Such polyclonal or monoclonal antibodies can be raised using the sequence of SEQ ID NO:2, or any immunogenic portion thereof.

HIV envelope glycoprotein strain variants and orthologs can be isolated using corresponding nucleic acid probes known in the art to screen libraries under stringent hybridization conditions. Alternatively, expression libraries can be used to clone sequences encoding HIV envelope glycoprotein strain variants and orthologs by detecting expressed proteins immunologically with commercially available antisera or antibodies, or antibodies made against SEQ ID NO:2, or portions thereof, which also recognize and selectively bind to the HIV envelope glycoprotein strain variants and orthologs.

To make a cDNA library, one should choose a source that is rich in the HIV envelope glycoprotein(s) of interest, such as the primary R5X4 HIV-1 isolate 89.6 described in Collman, R, et al. “An infectious molecular clone of an unusual macrophage-tropic and highly cytopathic strain of human immunodeficiency virus type 1”, J. Virol., 66, 7517-7521 (1992). The mRNA is then made into cDNA using reverse transcriptase, ligated into a recombinant vector, and transfected into a recombinant host for propagation, screening and cloning. Methods for making and screening cDNA libraries are well known (see, e.g., Gubler and Hoffman, Gene 25:263-269 (1983); Sambrook et al., supra; Ausubel et al., supra).

An alternative method of isolating nucleic acids encoding HIV envelope glycoproteins combines the use of synthetic oligonucleotide primers and amplification of an RNA or DNA template (see U.S. Pat. Nos. 4,683,195 and 4,683,202; PCR Protocols: A Guide to Methods and Applications (Innis et al., eds, 1990)). Methods such as polymerase chain reaction (PCR) and ligase chain reaction (LCR) can be used to amplify the nucleic acid sequences encoding the glycoproteins directly from mRNA, from cDNA present in genomic libraries or cDNA libraries. Degenerate oligonucleotides can be designed to amplify HIV envelope glycoproteins using the sequences provided herein. Restriction endonuclease sites can be incorporated into the primers. Polymerase chain reaction or other in vitro amplification methods may also be useful, for example, to clone nucleic acid sequences that code for proteins to be expressed, to make nucleic acids to use as probes for detecting the presence of HIV envelope glycoprotein-encoding mRNA in physiological samples, for nucleic acid sequencing, or for other purposes. Genes amplified by the PCR reaction can be purified from agarose gels and cloned into an appropriate vector.

HIV envelope glycoprotein gene expression can also be analyzed by techniques known in the art, e.g., reverse transcription and amplification of mRNA, isolation of total RNA or poly A⁺ RNA, northern blotting, dot blotting, in situ hybridization, RNase protection, high density polynucleotide array technology and the like.

Synthetic oligonucleotides can be used to construct recombinant HIV envelope glycoprotein genes for use as probes or for expression of protein. This method is performed using a series of overlapping oligonucleotides usually 40-120 bp in length, representing both the sense and non-sense (antisense) strands of the gene. These DNA fragments are then annealed, ligated and cloned. Alternatively, amplification techniques can be used with precise primers to amplify a specific gene subsequences for HIV envelope glycoproteins. The specific subsequence is then ligated into a suitable eukaryotic expression vector.

Whether comparing gp160/140, gp120 or gp41 homologues, DNA encoding HIV envelope glycoprotein strain variants and orthologs typically show at least 70% sequence identity between strains, as defined supra, and are capable of selectively cross-hybridizing when annealed under stringent hybridization conditions. Coding regions for field isolates of gp160/140, gp120 or gp41 will typically not vary in length by more than 6 base pairs.

HIV envelope glycoprotein genes can also be identified by reference to the proteins produced when expressed in a eukaryotic system. For example, a nucleic acid sequence or a restriction fragment putatively encoding gp160 can be inserted into a vector capable of transfecting a eukaryotic cell, providing a recombinant vector. The vector can then be used to transfect a eukaryotic cell capable of expressing the gp160 human immunodeficiency virus envelope protein. After culturing the recombinant mammalian cell under conditions suitable for expression of the recombinant HIV protein, the cell preparation can be tested for the presence of the HIV envelope using one of the protein-specific assays described infra.

Fusion Gene/Protein Construction

Polypeptide Linker Characteristics

The term “fusion protein” herein refers to the protein resulting from the expression of gp120 and gp41 operatively-linked coding sequences. These fusion proteins include constructs in which the C-terminal portion of gp120 is fused to the N-terminal portion of gp41 via an intervening in frame linker sequence.

Linkers are generally polypeptides of between 6 and 28 amino acids in length. The linkers joining the two molecules are preferably designed to allow the two molecules to fold and act independently of each other, not have a propensity for developing an ordered secondary structure which could interfere with the functional subunits of the two proteins, have minimal hydrophobic or charged characteristic which could interact with the functional protein subunits and prevent complete dissociation of gp120 from gp41 but still allow limited conformational changes that can lead to exposure of conserved epitopes able to elicit broadly cross-reactive HIV neutralizing antibodies.

Typically surface amino acids in flexible protein regions include Gly, Asn and Ser. Virtually any permutation of amino acid sequences containing Gly, Asn and Ser would be expected to satisfy the above criteria for a linker sequence. Other neutral amino acids, such as Thr and Ala, may also be used in the linker sequence. Preferably such neutral amino acids will have a relatively small surface area (160 A2, or less). Additional amino acids may also be included in the linkers due to the addition of unique restriction sites to facilitate construction of the fusions.

Exemplary linkers of the present invention include sequences selected from the group of formulas:

(GlySer)ₙ, (Gly₃Ser)ₙ, (Gly₄Ser)ₙ, (Gly₅Ser)ₙ,
(GlyₙSer)ₙ or (AlaGlySer)ₙ

where n can take a value with in the range 3 to 12. Additional examples of preferred linkers are set out in SEQ ID NOS:10 through 14.

The present invention is however, not limited by the form, size, composition or number of linker sequences employed. The only requirement of the linker is that, functionally, it does not interfere adversely with the folding and fuction of the individual molecules of the fusion, and otherwise allows for expression of the chimeric fusion molecule. One test of linker functionality is through inhibition of syncytia formation and reporter gene (β-gal and luciferase) assays described in detail in Example 2. Linker constructs of this invention form fusion proteins displaying at least 50% inhibition (at approx. 100 ng/ml fusion protein) by either assay. The fusion proteins also specifically bind antibodies raised against gp120 and gp41.

The present invention also includes linkers in which an endopeptidase recognition sequence is included. Such a cleavage site may be valuable to separate the individual components of the fusion to, for example, determine if they are properly folded and active in vitro. Examples of various endopeptidases include, but are not limited to, Plasmin, Enterokinase, Kallikrein, Urokinase, Tissue Plasminogen activator, clostripain, Chymosin, Collagenase, Russell's Viper Venom Protease, Postproline cleavage enzyme, V8 protease, Thrombin and factor Xa.

Construction from the gp160/140 Gene

Fusion proteins of the invention can also be produced from a full length gp160 coding sequence, or from a variant species of the gp160 gene, termed gp140, where the transmembrane subunit and the cytoplasmic tail of the gp41 subunit has been removed by nuclease treatment, or is simply altered in sequence by the introduction of stop codons preceding the transmembrane coding segment of gp41, preventing its translation (GenBank accession numbers U39362, AAA81043). The transmembrane subunit is defined as 3′ end of the gp160 gene sequence, beginning within 5 amino acids either side of residue 684 as noted in SEQ ID NO:7. Alternative sources of gp160 are known and include gene bank entries;
gi|18996245|emb|AJ417431.1|HIM417431[18996245];
gi|18996239|emb|AJ417428.1|HIM417428[18996239];
gi|18996233|emb|AJ417425.1|HIM417425[18996233];
gi|18996227|emb|AJ417422.1|HIM417422[18996227];
gi|18996221|emb|AJ417419.1|HIM417419[18996221]; and
gi|18996215|emb|AJ417416.1|HIM417416[18996215].

Regardless of which form or variant of the gp160 gene is used, a fusion protein between gp120 and gp41 joined by a flexible linker can be created by identical methodology known in the art (see Maniatis, Fritsch and Sambrook, Molecular Cloning: A Laboratory Manual, 2nd Ed. (1989); DNA Cloning: A Practical Approach, Volumes I and II (D. N. Glover ed. 1985); Current Protocols in Molecular Biology (Ausubel et al., eds., 1994)). For (KR to ID) are mutated in the region encoding the proteolytic cleavage site between gp120 and gp41. These mutations create two new restriction sites, EcoRI and EcoRV, which allows for the incorporation a polypeptide linker having ends of (GGSGG). Examples of the complete primary sequence of fusion proteins constructed in this manner are set out in SEQ ID NOS:7 and 8.

Construction from Separate gp120 and gp41 Genes

An alternative to producing the fusion proteins of the invention from a full length gp160 coding sequence involves assembling the fusion protein from independent component parts. gp120 and gp41 can be amplified from cDNA's produced by reverse transcription of the respective mRNA's. Alternatively, both proteins can be synthesized de novo by phosphoramidite chemistry commonly known in the art.

Numerous sequences for gp120 are known. See for example, Muesing et al., Nature 313:450458 (1985); Myers et al., “Human Retroviruses and AIDS; A compilation and analysis of nucleic acid and amino acid sequences,” Los Alamos National Laboratory, Los Alamos, N. Mex. (1992); McCutchan et al., AIDS Res. and Human Retroviruses 8:1887-1895 (1992); Gurgo et al., Virol. 164: 531-536 (1988).

The nucleotide sequence of DNA encoding gp120 or a relevant portion of gp120 can be determined and the amino acid sequence of gp120 can be deduced. Methods for amplifying gp120-encoding DNA from HIV isolates to provide sufficient DNA for sequencing are well known. In particular, Ou et al, Science 256:1165-1171 (1992); Zhang et al. AIDS 5:675-681 (1991); and Wolinsky, Science 255:1134-1137 (1992) describe methods for amplifying gp120 DNA Sequencing of the amplified DNA is well known and is described in Maniatis et al., Molecular Cloning—A Laboratory Manual, Cold Spring Harbor Laboratory (1984), and Horvath et al., An Automated DNA Synthesizer Employing Deoxynucleoside 3′-Phosphoramidites, Methods in Enzymology 154: 313-326 (1987), for example. In addition, automated instruments that sequence DNA are commercially available.

The nucleotide sequence encoding gp120 is present in an expression construct under the transcriptional and translational control of a promoter for expression of the encoded protein. The promoter can be a eukaryotic promoter for expression in a mammalian cell. In cases where one wishes to expand the promoter or produce gp120 in a prokaryotic host, the promoter can be a prokaryotic promoter. Usually a strong promoter is employed to provide high level transcription and expression.

Nucleotide sequences encoding gp41 are similarly common and can be recovered from any HIV isolate using for example labeled probes derived from SEQ IDs 1 or 3. gp41 coding sequences can also be isolated from known gp160 and gp140 sequences by molecular biological techniques known in the art, such as those described supra In this latter context, gp41 coding subunits used to construct the fusion proteins of this invention can be either the full-length form having the transmembrane subunit, or the truncated form-derived from the gp140 variant which lacks the coding sequence for the transmembrane subunit.

One of ordinary skill in the art will be able to adapt a linker to join independently amplified gp120 and gp41 coding sequences using routine PCR and other molecular biological techniques as described for example in Soo Hoo et al., PNAS 89:4759-4763 (1992) and Kim et al., Protein Engineering 2(8):571-575 (1989). Soo Hoo et al. discloses a linker connecting the variable regions of the α and β chains of a T cell receptor. Kim et al. discloses a linker designed to link the two polypeptide chains of monellin, a multi-chain protein known for its sweet taste.

The order in which the nucleic acids encoding the polypeptides are connected (carboxy-terminal end of gp120 is covalently linked through a peptide linker to the amino-terminal end of gp41) reflects the relationship of the polypeptides in their native state. Moreover, all of the nucleic acid components of the fusion are joined to produce a fusion product that is in frame.

Identifying Fusion Gene Sequences by Homology and Expression Product

Genes encoding the fusion protein can be identified by any of the techniques described above for nucleotide sequences encoding HIV envelope glycoproteins. In particular, it is useful to evaluate the nucleotide sequence encoding the fusion protein for the presence of both a gp120 subunit and a gp41 subunit. For example, fusion sequences will possess subunits with at least 70% homology to both gp120 and gp41, and will cross-hybridize with those coding sequences under stringent conditions. Fusion sequences can also be identified by the proteins that they produce. These proteins can be characterized by any of the methods used to characterize recombinant proteins described in detail below. Fusion sequences can also be identified by size, using for example agarose gel electrophoresis or differential filtration. Although the size of individual fusion proteins can vary as a consequence of slight variations in the strain-dependent size of the envelope components used, and both the linker length and sequence, the size of any given fusion sequence can be determined from the sum of the sizes of it's components determined as described in detail supra.

Expression of Fusion Proteins in Eukaryotic Cells

To obtain a high level of expression for a cloned gene, such as a cDNA encoding an HIV envelope glycoprotein, one typically subclones the gene into an expression vector that contains a strong promoter to direct transcription, operable 3′ end processing sequences, including a transcription/translation terminator, and a ribosome binding site for translational initiation. Eukaryotic expression systems for mammalian cells meeting these criteria are well known in the art and are commercially available. See Lasky et al., Science 233:209-212 (1986).

Selection of the promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be is accommodated without loss of promoter function.

In addition to the promoter, the expression vector typically contains a transcription unit or expression cassette that contains all the additional elements required for the expression of the HIV envelope glycoprotein encoding nucleic acid in host cells. A typical expression cassette thus contains a promoter operably linked to the nucleic acid sequence encoding the HIV envelope glycoproteins and signals required for efficient polyadenylation of the transcript, ribosome binding sites, and translation termination. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic cells may be used. Expression vectors containing regulatory elements from eukaryotic viruses are typically used in eukaryotic expression vectors, e.g., SV40 vectors, adenovirus, bovine papilloma virus, papilloma virus vectors, vectors derived from Epstein-Barr virus and the like. Other exemplary eukaryotic vectors include pLNSX, pMSG, pAV009/A⁺, pMTO10/A⁺, pMAMneo-5, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells. Expression constructs comprising coding sequences for the proteins of the present invention can be part of a vector capable of stable extrachromosomal maintenance in an appropriate cellular host or may be integrated into host genomes. Markers genes can be optionally included in the expression construct, allowing for selection of a host containing the construct. The marker can be on the same or a different DNA molecule, desirably, the same DNA molecule as the recombinant gene of the present invention. In addition, the construct may be joined to an amplifiable gene, e.g. DHFR gene, so that multiple copies of the gp120 DNA can be made.

Expression of proteins from eukaryotic vectors can be regulated using inducible promoters. With inducible promoters, expression levels are tied to the concentration of inducing agents, such as steriods or some metabolite, by the incorporation of response elements for these agents into the promoter. Generally, high level expression is obtained from inducible promoters only in the presence of the inducing agent. Inducible expression vectors are often chosen when expression of the protein of interest is detrimental to eukaryotic cells.

A preferred embodiment of the present invention comprises a constitutive promoter. Transcription from constitutive promoters is generally unaffected by inducing or repressing agents, and drive a constant, high rate of transcription. Promoters of the preferred embodiment should be particularly resistant to repression by cytokines, as cytokine production is stimulated by HIV envelope glycoproteins.

Standard transfection methods are used to produce mammalian cell lines that express large quantities of HIV envelope glycoproteins, which are then purified using standard techniques (see, e.g., Colley et al., J. Biol. Chem. 264:17619-17622 (1989); Guide to Protein Purification, in Methods in Enzymology, 182 (Deutscher, ed., 1990)). Transformation of eukaryotic are performed according to standard techniques (see, e.g., Clark-Curtiss and Curtiss, Methods in Enzymology 101:347-362 (Wu et al., eds, 1983)).

Any of the well-known procedures for introducing foreign nucleotide sequences into host cells may be used. These include the use of calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA or other foreign genetic material into a host cell. It is only necessary that the particular genetic engineering procedure used be capable of successfully introducing at least one gene into the host cell capable of expressing HIV envelope glycoproteins.

Preferably, HIV envelope glycoproteins are expressed in mammalian cells that provide the same glycosylation and disulfide bonds as in native envelope glycoproteins. Expression of gp120 and fragments of gp120 in mammalian cells as fusion proteins incorporating N-terminal sequences of Herpes Simplex Virus Type 1 (HSV-1) glycoprotein D (gD-1) is described in Lasky, L. A. et al. (Neutralization of the AIDS retroviris by antibodies to a recombinant envelope glycoprotein) Science 233:209-212 (1986) and Haffar et al. (The cytoplasmic tail of HIV-1 gp160 contains regions that associate with cellular membranes) Virol. 180:439-441 (1991), respectively. Examples of a mammalian cells capable of expressing the HIV envelope protein nucleic acid sequence as described here is the CEM cell line, available through the American Type Culture Collection (ATCC), and CHO cells as described in Berman et al., J. Virol. 66:4464-4469 (1992). Additional cell lines capable of expressing the fusion protein can be selected as described in Lasky et al., Science 223:209-212 (1986).

After the expression vector is introduced into the cells, the transfected cells are cultured under conditions favoring expression of the encoded recombinant HIV envelope glycoprotein, which is recovered from the culture using standard techniques identified below.

Purification and Identification of Fusion Proteins

Recombinant HIV envelope fusion proteins can be purified for use in functional assays, and can be purified from any suitable expression system. Recombinant HIV envelope fusion proteins may be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al., supra). A number of procedures can be employed to purify recombinant HIV envelope fusion proteins. For example, HIV envelope proteins could be purified using immunoaffinity columns, and have also been purified from growth-conditioned cell culture medium by immunoaffinity and ion exchange chromatography as described in Leonard et al., J. Biol. Chem. 265:10373-10382 (1990).

Solubility Fractionation

Often as an initial step, particularly if the protein mixture is complex, an initial salt fractionation can separate many of the unwanted host cell proteins (or proteins derived from the cell culture media) from the recombinant protein of interest The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

Size Differential Filtration

The molecular weight of the recombinant HIV envelope fusion proteins (e.g. in and around the range of 140 to 170 kDa) can be used to isolate it from proteins of greater and lesser size using ultrafiltration through membranes of different pore size (for example, Amicon or Millipore membranes). As a first step, the protein mixture is ultrfiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

Column Chromatography

The recombinant HIV envelope fusion proteins can also be separated from other proteins on the basis of size, net surface charge, hydrophobicity, and affinity for ligands. In addition, antibodies raised against proteins can be conjugated to column matrices and the proteins immunopurified. All of these methods are well known in the art It will be apparent to one of skill that chromatographic techniques can be performed at any scale and using equipment from many different manufacturers (e.g., Pharmacia Biotech or Merck).

Immunological Detection Of Recombinant HIV Envelope Fusion Proteins

In addition to the detection of recombinant HIV envelope fusion protein genes and gene expression using nucleic acid hybridization technology, one can also use immunoassays to detect the recombinant HIV envelope fusion proteins of the invention and to determine if an unknown protein is a protein of this invention. Immunoassays can be used to qualitatively or quantitatively analyze the recombinant HIV envelope fusion proteins. A general overview of the applicable technology can be found in Harlow and Lane, Antibodies: A Laboratory Manual (1988).

Antibodies to Recombinant HIV Envelope Fusion Proteins

Methods of producing polyclonal and monoclonal antibodies that react specifically with recombinant HIV envelope fusion proteins are known to those of skill in the art (see, e.g., Coligan, Current Protocols in Immunology (1991); Harlow and Lane, supra; Goding, Monoclonal Antibodies: Principles and Practice (2d ed. 1986); and Kohler and Milstein, Nature 256:495-497 (1975). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)).

A number of immunogens comprising portions of recombinant HIV envelope fusion proteins may be used to produce antibodies specifically reactive with recombinant HIV envelope fusion proteins. For example, recombinant HIV envelope fusion proteins or an antigenic fragment thereof can be isolated as described herein. Recombinant protein can be expressed in eukaryotic cells as described above, and purified as generally described above. Recombinant protein is the preferred immunogen for the production of monoclonal or polyclonal antibodies. Alternatively, a synthetic peptide derived from the sequences disclosed herein and conjugated to a carrier protein can be used an immunogen. Naturally occurring protein may also be used either in pure or impure form. The product is then injected into an animal capable of producing antibodies. Either monoclonal or polyclonal antibodies may be generated, for subsequent use in immunoassays to measure the protein.

Methods of production of polyclonal antibodies are known to those of skill in the art. An inbred strain of mice (e.g., BALB/C mice) or rabbits is immunized with the protein using a standard adjuvant, such as Freund's adjuvant, and a standard immunization protocol. The animal's immune response to the immunogen preparation is monitored by taking test bleeds and determining the titer of reactivity to the beta subunits. When appropriately high titers of antibody to the immunogen are obtained, blood is collected from the animal and antisera are prepared. Further fractionation of the antisera to enrich for antibodies reactive to the protein can be done if desired (see, Harlow & Lane, supra).

Monoclonal antibodies may be obtained by various techniques familiar to those skilled in the art. Briefly, spleen cells from an animal immunized with a desired antigen are immortalized, commonly by fusion with a myeloma cell (see, Kohler and Milstein, Eur. J. Immunol. 6:511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse, et al., Science 246:1275-1281 (1989).

Monoclonal antibodies and polyclonal sera are collected and titered against the immunogen protein in an immunoassay, for example, a solid phase immunoassay with the immunogen immobilized on a solid support Typically, polyclonal antisera with a titer of 10⁴ or greater are selected and tested for their cross reactivity against non-HIV envelope proteins, using a competitive binding immunoassay. Specific polyclonal antisera and monoclonal antibodies will usually bind with a K_{d} of at least about 0.1 mM, more usually at least about 1 μM, preferably at least about 0.1 μM or better, and most preferably, 0.01 μM or better. Once the specific antibodies against HIV envelope proteins are available, the recombinant HIV envelope fusion proteins can be detected by a variety of immunoassay methods. For a review of immunological and immunoassay procedures, see Basic and Clinical Immunology (Stites & Terr eds., 7^{th} ed. 1991). Moreover, the immunoassays of the present invention can be performed in any of several configurations, which are reviewed extensively in Enzyme Immunoassay (Maggio, ed., 1980); and Harlow and Lane, supra.

Immunological Binding Assays

The recombinant HIV envelope fusion proteins of the invention can be detected and/or quantified using any of a number of well recognized immunological binding assays (see, e.g., U.S. Pat. Nos. 4,366,241; 4,376,110; 4,517,288; and 4,837,168). For a review of the general immunoassays, see also Methods in Cell Biology: Antibodies in Cell Biology, volume 37 (Asai, ed. 1993); Basic and Clinical Immunology (Stites and Terr, eds., 7^{th} ed. 1991). Immunological binding assays (or immunoassays) typically use an antibody that specifically binds to a protein or antigen of choice (in this case the HIV envelope fusion proteins or an antigenic subsequence thereof). The antibody (e.g., anti-HIV envelope protein) may be produced by any of a number of means well known to those of skill in the art and as described above.

Immunoassays also often use a labeling agent to specifically bind to and label the complex formed by the antibody and antigen. The labeling agent may itself be one of the moieties comprising the antibody/antigen complex. Thus, the labeling agent may be a labeled polypeptide derived from an HIV envelope protein or a labeled anti-HIV envelope protein antibody. Alternatively, the labeling agent may be a third moiety, such a secondary antibody, which specifically binds to the antibody/HIV envelope fusion protein complex (a secondary antibody is typically specific to antibodies of the species from which the first antibody is derived). Other proteins capable of specifically binding immunoglobulin constant regions, such as protein A or protein G may also be used as the label agent. These proteins exhibit a strong non-immunogenic reactivity with immunoglobulin constant regions from a variety of species (see, e.g., Kronval et al., J. Immunol. 111: 1401-1406 (1973); Akerstrom et al., J. Immunol. 135:2589-2542 (1985)). The labeling agent can be modified with a detectable moiety, such as biotin, to which another molecule can specifically bind, such as streptavidin. A variety of detectable moieties are well known to those skilled in the art.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 1° C. to 40° C.

Non-Competitive Assay Formats

Immunoassays for detecting recombinant HIV envelope fusion proteins in samples may be either competitive or noncompetitive. Noncompetitive immunoassays are assays in which the amount of antigen is directly measured. In one preferred “sandwich” assay, for example, the anti-HIV envelope proteins antibodies can be bound directly to a solid substrate on which they are immobilized. These immobilized antibodies then capture recombinant HIV envelope fusion proteins present in the test sample. The recombinant HIV envelope fusion proteins thus immobilized are then bound by a labeling agent, such as a second HIV envelope protein antibody bearing a label. Alternatively, the second antibody may lack a label, but it may, in turn, be bound by a labeled third antibody specific to antibodies of the species from which the second antibody is derived. The second or third antibody is typically modified with a detectable moiety, such as biotin, to which another molecule specifically binds, e.g., streptavidin, to provide a detectable moiety.

Competitive Assay Formats

In competitive assays, the amount of the recombinant HIV envelope fusion protein present in the sample is measured indirectly by measuring the amount of known, added (exogenous) envelope fusion protein displaced (competed away) from an anti-HIV envelope protein antibody by the unknown amount of recombinant HIV envelope fusion protein present in the sample. In one competitive assay, a known amount of the HIV envelope protein is added to a sample and the sample is then contacted with an antibody that specifically binds to the envelope protein. The amount of exogenous envelope protein bound to the antibody is inversely proportional to the concentration of the envelope protein present in the sample. In a particularly preferred embodiment, the antibody is immobilized on a solid substrate. The amount of envelope fusion protein bound to the antibody may be determined either by measuring the amount of envelope protein present in a antibody/envelope fusion protein complex, or alternatively by measuring the amount of remaining uncomplexed protein. The amount of envelope fusion protein may be detected by providing a labeled envelope fusion protein molecule.

A hapten inhibition assay is another preferred competitive assay. In this assay envelope fusion protein is immobilized on a solid substrate. A known amount of anti-envelope protein antibody is added to the sample, and the sample is then contacted with the immobilized envelope fusion protein. The amount of anti-envelope protein antibody bound to the known immobilized envelope fusion protein is inversely proportional to the amount of envelope fusion protein present in the sample. Again, the amount of immobilized antibody may be detected by detecting either the immobilized fraction of antibody or the fraction of the antibody that remains in solution. Detection may be direct where the antibody is labeled or indirect by the subsequent addition of a labeled moiety that specifically binds to the antibody as described above.

Affinity Purification Determinations

Affinity purification of a polyclonal antibody pool or sera provides a practitioner with a more uniform reagent for conducting immunological screens and identifications, including those presented here by way of example. Briefly, a polyclonal antibody pool or sera obtained from an individual inoculated with envelope fusion protein can be used to select out anti-envelope fusion protein antibodies. Such methods are well known in the art and available commercially (AntibodyShop, c/o Statens Serum Institut, Artillerivej 5, Bldg. P2, DK-2300 Copenhagen S). Briefly, envelope fusion protein is attached to an affinity support (see e.g.; CNBR Sepharose (R), Pharmacia Biotech) and used to form an afinnity column. The polyclonal antibody pool or sera is then passed down the affinity column. Antibodies in the polyclonal pool which recognize the envelope fusion protein bind to the column, the remainder passing through. Bound antibodies are then released by techniques common to those familiar with the art, yielding an antibody pool highly enriched for antibodies recognizing envelope fusion protein epitopes. This enriched anti-envelope fusion protein antibody pool can then be used for further immunological studies, some of which are described herein by way of example.

For example, the enriched anti-envelope fusion protein antibody pool can be used in a competitive binding immunoassay as described above to compare a second protein, thought to be perhaps a variant of the HIV envelope protein of this invention. In order to make this comparison, the two proteins are each assayed at a wide range of concentrations and the amount of each protein required to inhibit 50% of the binding of the antisera to the immobilized protein is determined. If the amount of the second protein required to inhibit 50% of binding is less than 10 times the amount of envelope fusion glycoprotein required to inhibit 50% of binding, then the second protein is said to specifically bind to the polyclonal antibodies generated to the respective envelope fusion glycoprotein immunogen.

Western Blotting

Additional analyses of specificity were carried out by Western blot (Biotech Research Labs, Rockville, Md. and Immunetics, Cambridge, Mass.). The technique generally comprises separating sample proteins by gel electrophoresis on the basis of molecular weight, transferring the separated proteins to a suitable solid support (such as a nitrocellulose filter, a nylon filter, or derivatized nylon filter), and incubating the sample with the antibodies that recognize fusion glycoprotein-antigen from primary viral isolates, infected T-cells, or the like, according to standard techniques. The anti-fusion glycoprotein-antibodies specifically bind to fusion glycoprotein immobilized on the solid support. These antibodies may be directly labeled or alternatively may be subsequently detected using labeled antibodies (e.g., labeled sheep anti-mouse antibodies) that specifically bind to the fusion glycoprotein antibodies.

Other assay formats include liposome immunoassays (LIA), which use liposomes designed to bind specific molecules (e.g., antibodies) and release encapsulated reagents or markers. The released chemicals are then detected according to standard techniques (see, Monroe et al., Amer. Clin. Prod. Rev. 5:3441 (1986)).

Reduction of Non-Specific Binding

One of skill in the art will appreciate that it is often desirable to minimize non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred.

Labels

The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., DYNABEADS™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic beads (e.g., polystyrene, polypropylene, latex, etc.).

The label may be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to another molecule (e.g., streptavidin), which is either inherently detectable or covalently bound to a signal system, such as a detectable enzyme, a fluorescent compound, or a chemiluminescent compound. The ligands and their targets can be used in any suitable combination with antibodies that recognize recombinant HIV envelope fusion proteins, or secondary antibodies that recognize anti-HIV envelope protein antibodies.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see, U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally, simple colorimetric labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

Sources of Antibodies

In addition to preparation and purification of antibodies de novo according to the methods noted supra, anti-HIV envelope glycoprotein antibodies are also commercially available. For example, unconjugated goat anti-gp41 (Cat #1971) and anti-gp120 (Cat# 1961) antibodies are available from ViroStat P.O. Box 8522, Portland, Me. 04104. The same antibodies are also available in several pre-labeled varieties (gp41: Biotinylated (Cat# 1977), FITC (Cat # 1973), or HRP (Cat #1974). gp120: Biotinylated (Cat # 1961), FITC (Cat# 1963), or HRP (Cat #1964)). Other commercial sources include Trinity Biotech Plc, IDA Business Park, Bray, Co Wicklow, Ireland (anti-gp120 cat# 1001, anti-gp41 cat#1201); and Protein Sciences Corporation, 1000 Research Parkway, Meriden, Conn. 06450 (anti-gp 160 cat# 2000LAV, anti-gp120 cat# 2003LAV).

Vaccine Preparation and Use

Polypeptide Vaccines

Peptides of the present invention can elicit an immune response. Consequently, these peptides have use in a vaccine preparation against AIDS and AIDS related conditions. Immunogenic compositions containing proteins and complexes of the invention and suitable for use as a vaccine, elicit an immune response which produces antibodies that are opsonizing or antiviral. Should the vaccinated subject be challenged by HIV, the antibodies bind to the virus and thereby neutralize it.

Formulation

Vaccines containing peptides are generally well known in the art, as exemplified by U.S. Pat. Nos. 6,080,570; 6,107,021; 6,248,582; and 6,342,224. Vaccines may be prepared as injectables, as liquid solutions or emulsions. The peptides may be mixed with pharmaceutically-acceptable excipients which are compatible with the peptides and are nontoxic to a recipient at the dosage and concentration employed in the vaccine. Excipients may include water, saline, dextrose, glycerol, ethanol, and combinations thereof. Vaccines may be administered parenterally, by injection subcutaneously or intramuscularly. Alternatively, other modes of administration including suppositories and oral formulations may be desirable. For suppositories, binders and carriers may include, for example, polyalkalene glycols or triglycerides. Oral formulations may include normally employed incipients such as, for example, pharmaceutical grades of saccharine, cellulose and magnesium carbonate. These compositions take the form of solutions, suspensions, tablets, pills, capsules, sustained release formulations or powders and contain 5-98% of the peptides.

The vaccine may further contain auxiliary substances such as wetting or emulsifying agents, pH buffering agents, chelating agents, or adjuvants to enhance the effectiveness of the vaccines. Methods of achieving adjuvant effect for the vaccine include the use of agents such as aluminum hydroxide or phosphate (alum), commonly used as 0.05 to 0.1 percent solution in phosphate buffered saline or QS21 which stimulates cytotoxic T-ells. Formulations with different adjuvants which enhance cellular or local immunity can also be used. The relative proportion of adjuvant to immunogen can be varied over a broad range so long as both are present in effective amounts. For example, aluminum hydroxide can be present in an amount of about 0.5% of the vaccine mixture (Al2O3 basis).

Peptide vaccine preparations of the present invention can be further augmented by addition of soluble binding subunits derived from natural gp160 receptors, such as CD4, CCR5 and CXCR4. These additional soluble binding subunits can be incorporated into the vaccine as separate peptides which interact with the gp120/gp41 fusion protein via the purely non-covalent interactions, normal to this receptor/ligand complex. Alternatively, the soluble binding subunits can be covalently bound to the gp120/gp41 fusion protein via a peptide linker. In this latter format, the linker performs an identical function to the linker tethering gp120 to gp41, the purpose being to retain proximity of the molecules, thereby inducing them to interact in a normal receptor/ligand complex such that the half-life of the complex is prolonged. Through formation of the receptor/ligand complex, both the receptor and ligand can undergo conformational changes exposing epitopes hidden in the isolated molecules. These “conformationally induced” epitopes include epitopes capable of generating immune responses against the HIV envelope glycoprotein not normally occurring in the absence of complex formation with the HIV envelope protein. Dimitrov, D. S., Cell 101(7):697-702 (2000).

Conveniently, the vaccines are formulated to contain a final concentration of immunogen in the range from 0.2 to 200 μg/ml, preferably 5 to 50 μg/ml, most preferably 15 g/ml. After formulation, the vaccine may be incorporated into a sterile container which is then sealed and stored at a low temperature, for example 4° C. or it may be freeze-dried. Lyophilization permits long-term storage in a stabilized form.

Administration

The vaccines are administered in a manner compatible with the dosage formulation, and in such amount as is therapeutically effective and protective. Following the immunization procedure, annual or bi-annual boosts can be administered. During the immunization process and thereafter, neutralizing antibody levels can be assayed and the protocol adjusted accordingly. The quantity of vaccine administered however depends on the subject to be treated, including, for example, the capacity of the individual's immune system to synthesize antibodies, and to produce a cell-mediated immune response. The size of the active ingredient aliquot administered ultimately depends on the judgment of the practitioner. Suitable dosage ranges are however readily determinable by one skilled in the art and may be of the order of micrograms of the peptides. Suitable regimes for initial administration and booster doses are also variable, with the vaccine generally being administered as individual aliquots at 0, 1, and at 6, 8 or 12 months, depending on the protocol. An alternative protocol may include an initial administration followed by subsequent administrations, for example, at least one pre-peptide immunization with an aliquot comprising a self-assembled, non-infectious, non-replicating HIV-like particle, followed by at least one secondary immunization with an aliquot of the peptides provided herein The dosage of the vaccine may also depend on the route of administration and will vary according to the size of the host. On a per-dose basis, the amount of the immunogen can range from about 5 μg to about 200 μgprotein per inoculation. A preferable range is from about 20 μg to about 120 μg per dose. A suitable dose size is about 0.5 ml. Accordingly, a dose for intramuscular injection, for example, would comprise 0.5 ml containing 90 μg of immunogen in a mixture with 0.5% aluminum hydroxide administered to a healthy, HIV serial-negative individual of average weight (75 kg). Preferably, the vaccination protocol will be the same as protocols now used in clinical vaccination studies and disclosed in, for example, Reuben et al., J Acquired Immune Deficiency Syndrome, 5:719-725 (1992), incorporated herein by reference.

The use of the fusion proteins and complexes provided herein may require modification as the peptides themselves may not have a sufficiently long in-vivo serum and/or tissue half-life. For this purpose, the molecule of the invention may optionally be linked to a carrier molecule, possibly via chemical groups of amino acids of the conserved sequence or via additional amino acids added at the C- or N-terminus. Many suitable linkages are known, e.g., using the side chains of Tyr residues. Suitable carriers include, e.g., keyhole limpet hemocyanin (KLH), serum albumin, purified protein derivative of tuberculin (PPD), ovalbumin, non-protein carriers and many others.

DNA Vaccines

Nucleic acid molecules encoding the peptides of the present invention may also be used for immunization by direct administration of the nucleic acid, or by incorporating the nucleic acid into a live vector followed by administering the vector to a patient. Such vectors are typically in the form of a viral expression system (e.g., vaccinia or other pox virus, retrovirus, or adenovirus), which may involve the use of a non-pathogenic (defective), replication competent virus. Techniques for incorporating DNA into such expression systems are well known to those of ordinary skill in the art, for example, Fisher-Hoch et al., PNAS 86:317-321 (1989); Flexner et al., Ann. N Y Acad. Sci. 569:86-103 (1989); Flexner et al., Vaccine 8:17-21 (1990); U.S. Pat. Nos. 4,603,112, 4,769,330, 5,017,487, and 6,228,844; WO 89/01973; U.S. Pat. No. 4,777,127; GB 2,200,651; EP 0,345,242; WO 91/02805; Berkner, Biotechniques 6:61&627 (1988); Rosenfeld et al., Science. 252:431-434 (1991); Kolls et al., PNAS 91:215-219 (1994); Kass-Eisler et al., PNAS 90:11498-11502 (1993); Guzman et al., Circulation 88:2838-2848 (1993); O'Hagan, Clin. Phannokinet 22:1(1992); and Guzman et al., Cir. Res. 73:1202-1207 (1993). When incorporated into expression systems, the nucleic acid construct contains the necessary regulatory and induction sequences for expression of the immunogenic DNA in the patient (such as a suitable promoter).

Nucleic acid administered directly is termed “naked” DNA, and has been described, for example, in published PCT application WO 90/11092, and Ulmer et al., Science 259:1745-1749 (1993), reviewed by Cohen, Science 259:1691-1692 (1993) and Ulmer et al, Curr. Opinion Invest. Drugs 2(9):983-989 (1993). Naked DNA can be injected into muscle or other tissue subcutaneously, intradermally, intravenously, or may be taken orally or directly into the spinal fluid. Of particular interest is injection into skeletal muscle. An example of intramuscular injection may be found in Wolff et al., Science 247:1465-1468 (1990). Jet injection may also be used for intramuscular administration, as described by Furth et al., Anal Biochem 205:365-368 (1992). The DNA may be coated onto gold microparticles, and delivered intradermally by a particle bombardment device, or “gene gun”. Microparticle DNA vaccination has been described in the literature (see, for example, Tang et al. Nature 356:152-154 (1992)). Alternatively, the naked DNA may be coated onto biodegradable beads, which are efficiently transported into the cells.

In general, the dose of a naked nucleic acid composition such as a DNA vaccine or gene therapy vector is from about 1 μg to 100 μg for a typical 70 kilogram patient. The immunogenic composition can be either a nucleic acid encoding the target protein (e.g., a DNA vaccine) or a virus vector which produces the antigenic protein. Subcutaneous or intramuscular doses for naked nucleic acid (typically DNA encoding a fusion protein) will range from 0.1 μg to 500 μg for a 70 kg patient in generally good health. Subcutaneous or intramuscular doses for viral vectors comprising the fusion proteins of the invention will range from 105 to 109 pfu for a 70 kg patient in generally good health.

Alternative uses for Fusion Proteins and Complexes of the Invention and Antibodies to the Same

The fusion proteins, complexes, or antibodies thereto can also be used in a method for the detection of HIV infection. For instance the complex, which is bound to a solid substrate or labeled, is contacted with the test fluid and immune complexes formed between the complex of the present invention and antibodies in the test fluid are detected. Preferably, antibodies raised against the immunogenic complexes of the present invention are used in a method for the detection of HIV infection. These antibodies may be bound to a solid support or labeled in accordance with known methods in the art. The detection method would comprise contacting the test fluid with the antibody and immune complexes formed between the antibody and antigen in the test fluid are detected and from this the presence of HIV infection is determined. The immunochemical reaction which takes place using these detection methods is preferably a sandwich reaction, an agglutination reaction, a competition reaction or an inhibition reaction.

As the fusion proteins in accordance with the present invention have HIV chemopreventative properties, they could also be utilized as part of a prophylactic regimen designed to prevent, or protect against, possible HIV infection upon sexual contact with an infected individual. In this sense, one or more proteins or complexes of the invention may also be formulated into a creme, lotion, douche or into the lining of a condom. The preparation of such cremes, lotions and douches will also be generally known to those of skill in the art.

Chemopreventative vaginal douche and cremes containing the proteins and complexes of the invention may be of use in connection with pre-sexual exposure protection. Such douches and cremes may be formulated in a standard acetic acid solution. The cremes may also be mixed with 9-nonoxynol spermicide to use in conjunction with birth control, or added to condoms. Vaginal sponges containing the peptides form another aspect of the invention, in such cases, the active peptides or agents may be time-released over several hours with nonoxynol.

The proteins and complexes may also be formulated in suppository forms for use in connection with chemoprevention during anal sex, because the rectum and large intestine are major sites of HIV infection. In the prevention of oral sex contraction of HIV, the mixing of the proteins and complexes in slippery oils that taste good (i.e. Motion Lotion or Blow Hot Oil) is also contemplated.

The proteins and complexes may be used in their chemopreventative capacity be administering in an amount that is effective in a preventative manner. In this sense, an “effective preventative amount” means an amount of composition that contains an amount of a fusion protein of complex sufficient to significantly inhibit or prevent HIV infection of cells in an uninfected animal on contact with an infected animal. If required, for example by insurance companies, the fusion proteins or complexes may also be added to gloves used by health care workers or researchers dealing heavily with blood and bodily fluids or to liquid soap used in hospitals and research institutions.

In addition to screening antibodies with a anti-fusion protein antibody, random or combinatorial peptide libraries can be screened with either an anti-fusion protein antibody or the fusion proteins or complexes of the invention. Approaches are available for identifying peptide ligands from libraries that comprise large collections of peptides, ranging from 1 million to 1 billion difference sequences, which can be screened using monoclonal antibodies or target molecules. The power of this technology stems from the chemical diversity of the amino acids coupled with the large number of sequences in a library. See for example, Scott et al., Cur. Open. Biotechnol. 5(1):40-8 (1994); Kenan et al. Trends Biochem. Sci. 19(2):57-64 (1994). Accordingly, the monoclonal antibodies, preferably human monoclonal antibodies, or fragments thereof, generated as discussed herein, find use in treatment by inhibiting or treating HIV infection or disease progression, as well as in screening assays to identify additional pharmaceuticals.

A further and important use of the anti-idiotope antibodies described herein concerns their attachment to solid supports and columns, such as Sepharose and agarose columns, sterile HPLC resins, and the like. Such supports and columns with appended peptides, e.g., HIV envelope glycoprotein affinity columns, may be used for inactivating HIV from within blood and other body fluid samples. One particular use would then be as disposable filters for deactivating HIV within blood and blood by-products.

### EXAMPLES

The following examples are offered to illustrate, but not to limit the claimed invention.

### Example 1

### Construction of Expression Constructs for the Tethered HIV-189.6 and the Isolation and Characterization of the Fusion Proteins

Stable fusion proteins of gp120 and gp41 joined by flexible linkers were created using the envelope glycoprotein from the primary R5X4HIV-1 isolate 89.6 as starting material. Two amino acid residues of the post translational cleavage site REKR, were mutated by PCR changing the sequence of the site to REID. Two new restriction sites, EcoRI and EcoRV, were introduced into the sequence. Introduction of the restriction sites created a short fragment (EFIS) following the mutated cleavage site. Flexible linkers were introduced into the middle of this sequence by PCR. Three different fusion proteins where gp120 andgp41 are joined by fragments of different total lengths 4 (SEQ ID NO:9), 15 (SEQ ID NO:10) or 26 (SEQ ID NO:11) amino acid residues were developed. Using the same technique, a stop codon was introduced at position 668 of the env protein sequence (GenBank accession numbers U39362, AAA81043) and three additional amino acids (KLV) added at the very end of the linker proteins. The stop codons result in proteins that are truncated N-terminal to the transmembrane domain of gp41. Since the fusion proteins do not contain the transmembrane domain and cytoplasmic tail of gp41, they are secreted in the medium of the expressing cells. Thus, three different fusion proteins, designated gp140-4, gp140-15 and gp140-26 were developed.

The fusion proteins were introduced into plasmid pEF1/His and the final constructs were used to transfect 293T cells. 293T cells were either stably or transiently transfected with the resulting expression vectors. Supernatant was collected from the stable transfectants or from the transiently transfected cell lines 48 hours after transfection. The supernatant was analysed for expression of the constructs by western blot using anti-gp120 and anti-gp41 antibodies.

Proteins were purified from the supernatant using lentil lectin Sepharose 4B affinity chromatography (Amersham-Pharmacia Biotech). Bound protein was eluted from the lectin column with 1M methyl-a-D-mannopyranoside. The eluted protein was dialyzed against PBS.

Purified fusion proteins were run on a 10% SDS-PAGE gel with calibrating amounts (1, 3, 10, 30, 100 ng) of highly purified gp140, and were electrophoretically transferred to nitrocellulose membranes. Membranes were blocked with 20 mM tris-HCl (pH 7.6) buffer containing 140 mM NaCl, 0.1% Tween-20 and 5% nonfat powdered milk. Membranes were incubated with anti gp120 antibodies, washed, then incubated with horseradish peroxidase (HRP)-conjugated secondary antibodies. Western blots were developed with supersignal chemiluminescent substrate from Pierce (Rockford, Ill.). Images were acquired using a BioRad phosphoimager (BioRad, Hercules, Calif.).

Concentration in the culture supernatants was about 5 μg/ml and after purification was about 0.7 mg/ml. The molecular weight (MW) of the fusion proteins on SDS PAGE was close to 140 kDa.

Size exclusion chromatography. The fusion proteins were analyzed under nondenaturing conditions by gel filtration chromatography on a preparative superdex200 column (Amersham-Pharmacia Biotech). The column was equilibrated with PBS, calibrated, and then standardized using protein standards ranging from 158 to 669 kDa. Samples of the fusion proteins were applied to the column in 1 ml of PBS. The column was run at a constant flow rate of approximately 1.1 ml/min, washed with PBS and fractions were collected. Size exclusion chromatography of the purified proteins revealed that they were predominantly monomeric with a very low concentration of dimers and gp120.

Flow cytometry cell surface binding assay. To determine whether the fusion proteins preserved their ability to bind their natural receptors, CD4 and CCR5, complexes of the fusion proteins with soluble CD4 (sCD4) were tested for their binding activity to native CCR5. Binding was measured by flow cytometry cell surface binding assay. Cells (typically.5×10⁶) were incubated for 1 h on ice with the fusion proteins and soluble CD4, then washed and incubated with gp120, CD4 or CCR5-specific antibodies at 1 μg/ml. Cells were washed, and incubated for another hour on ice with rabbit IgG (10 μg/ml) (Sigma, St. Louis, Mo.), then washed and incubated for 1 h with an anti-mouse phycoerythrin-conjugated polyclonal antibody or anti-rabbit FITC-conjugated polyclonal antibody for gp120 and CD4 (Sigma). Cells were washed and fixed with paraformaldehyde. Flow cytometry measurements were performed with FACS Calibur (Becton Dickinson, San Jose, Calif.). Results are shown in Table 1 below.

TABLE 1
Binding of gp140-15 complexed with two-domain sCD4 to cell surface
associated CCR5. CF2Th-CCR5 cells were incubated with
gp140-15, sCD4, gp140-15-sCD4, gp140-sCD4 at 5 μg/ml
(except soluble CD4 which was at 1 μg/ml) or without ligands at 4° C. for
1 h. Cell surface binding was tested by anti-CC5 mAb (5C7), anti-CD4
polyclonal antibody (T4-4) and an anti-gp120 polyclonal antibody (R2143)
using flow cytometry. The background binding was measured by
using the secondary antibody in the absence of the specific antibody
and subtracted. The binding is represented as the geometric mean
of fluorescence intensity in arbitrary units.

| Antibody/ | | | | gp140-15 + | gp140 + |
|---|---|---|---|---|---|
| Ligand | No ligand | sCD4 | gp140-15 | sCD4 | sCD4 |
| CCR5 | 1.1 × 103 | 1.3 × 103 | 1.2 × 103 | 1.1 × 103 | 370 |
| CD4 | 0.1 | 20 | 1 | 8 | 7 |
| gp120 | 0 | 0.02 | 1.3 | 20 | 5 |

These data demonstrate that the fusion binds well to CCR5 in the presence of CD4.

ELISA binding assay. To determine if the tethered fusion proteins are able to interact with receptors involved in HIV-1 cell entry, binding of purified molecules was tested using a modified (enzyme-linked immunosorbant assay) ELISA assay. The test proteins, e.g. soluble CD4, were non-specifically attached to the bottom of 96-well plates by incubation of 0.1 ml solution containing 100 ng of the protein at 4° C. overnight. To prevent nonspecific binding, plates were treated with PBS containing 2% BSA and 0.5. % Tween-20 (PBS-BSA-Tween). Plates were washed with TBS, test samples were diluted in PBS-BSA-Tween and incubated for 1 h at room temperature. Bound antigen was detected with anti-gp41 antibodies and the appropriate labeled secondary antibody. Biotinylated proteins for use in this assay were prepared by incubation with 2 mM biotin on wet ice for 1 h. The biotinylation was quenched with 20 mM glycine on ice for 15 min.

The tethered proteins complexed with two-domain soluble CD4 (sCD4) bound cell surface-associated CCR5 similarly to uncleaved gp140 complexed with sCD4. There was no binding of sCD4, gp140-15-sCD4 or the anti-CCR5 mAb 5C7 to the parental cell line Cf2Th. These data suggest that the expressed fusion proteins are able to interact with receptors involved in HIV-1 entry.

The native conformation of the tethered proteins was also tested by ELISA using conformationally dependent anti-gp120 (M12 and D25) and anti-gp41 (D54) mAbs. There were no significant differences in the binding of these antibodies to gp140-4, 15, 26 compared to uncleaved gp140. These data suggest that the tethered proteins are likely to be antigenically similar to uncleaved Envs.

### EXAMPLE 2

### Using Tethered HIV-1 Envelope Glycoproteins to Inhibit Cell Fusion and Virion Entry into Cells

Cell-cell fusion. To determine if the tethered gp140s inhibit Env-mediated membrane fusion, a β-gal reporter gene and syncytia formation assays for cell fusion were performed (Table 2). Briefly, in the cell-cell fusion assay, two cell types are mixed with the tethered envelope glycoprotein construct. One cell type expresses the T7 RNA polymerase. The second cell type contains the Beta-galactosidase gene under control of the T7 promoter. When fusion of the two cell types occurs, expression of Beta-galactosidase can be detected. The method is described in detail in Nussbaum et al., J. Virol. 68:5411-5422 (1994). Briefly, recombinant vaccinia viruses at multiplicity of infection 10 were used to infect the target (vCB21R) and effector cells (vTF 7.3) The beta-gal fusion assay was performed two hours after mixing the cells. The extent of fusion was quantitated colorimetrically.

Inhibition of cell-cell fusion was also quantitated by using a syncytium assay where cells expressing Env were mixed with equal number of cells expressing CD4 and coreceptor molecules, and the number of syncytia was counted 4 h later. Syncitia were counted microscopically as giant cells with a diameter larger than 2-3 cell diameters.

TABLE 2
Inhibition of cell fusion by gp140-15 and gp14-26. 10⁵ TF228 cells expressing
LAI Env and 10⁵ SupT1 cells were preincubated at different concentrations of the inhibitor
for 1 h at 37° C., then mixed together in a 96-well plate and incubated for 2 h at 37° C. followed
by measurement of β-gal activity or number of syncytia. The data are presented as
percentage of fusion in the absence of inhibitor, which is assumed to be 100%. The mean +/− standard
deviation of duplicate experiments is also given.

| Inhibitor | | | | | | |
|---|---|---|---|---|---|---|
| Concentration | sCD4 | gp140 | gp140-4 | gp140-26 | gp140-15 | gp140-15 |
| μg/ml | β-gal act. % | β-gal act. % | β-gal act. % | β-gal act. % | β-gal act. % | Syncytia % |
| 0.01 | 98 +/− 2 | 102 +/− 2 | 99 +/− 4 | 70 +/− 10 | 77 +/− 9 | 64 +/− 5^{a} |
| 0.1 | 95 +/− 4 | 98 +/− 3 | 100 +/− 2 | 24 +/− 1 | 39 +/− 1 | 29 +/− 16 |
| 1 | 67 +/− 1 | 100 +/− 2 | 98 +/− 1 | 15 +/− 1 | 22 +/− 4 | 11 +/− 6 |
| 10 | 25 +/− 2 | 77 +/− 5 | 76 +/− 7 | 8.2 +/− 0.5 | 14 +/− 1 | 7.4 +/− 2.1 |

One can see from the above Table that at 10 nM concentration, th gp140-26 and the gp140-15 constructs acted as potent inhibitors of cell-cell fusion. Thus they are effective HIV-1 cell entry inhibitors.

Inhibition of HIV-1 Env-mediated membrane fusion. To demonstrate any functional activity of the tethered proteins different than binding to receptor molecules we used an entry assay as a test system. Inhibitory activity would mean that they exhibit some structures that are able to interfere with entry by a mechanism different than direct binding to receptors. These structures could be used as immunogens for elicitation of neutralizing antibodies. Evaluation of HIV-1 entry inhibition was performed by using infection with a luciferase reporter HIV-1 Env pseudotyping system. The method is described in detail in Wild et al. Proc. Natl. Acad. Sci. U.S.A. 91:9770-9774 (1994).

Viral stocks were prepared by transfecting 293T cells with plasmids encoding the luciferase virus backbone (pNL-Luc-ER) and Env from various HIV strains. The resulting supernatant was clarified by centrifigation. The virus was preincubated with various concentrations of inhibitors for 1 h at 37° C. Cells were then infected with 100 μl of virus preparation containing DEAE-dextran (81 g/ml) for 4h at 37° C. Cells were washed and 0.2 ml was added to each well in a 96-well plate. Cells were lysed 44 h later by resuspension in 100 μl of cell lysis buffer (Promega, Madison, Wis.). 501 μl of the resulting lysate was assayed for luciferase activity, using an equal volume of luciferase substrate (Promega).

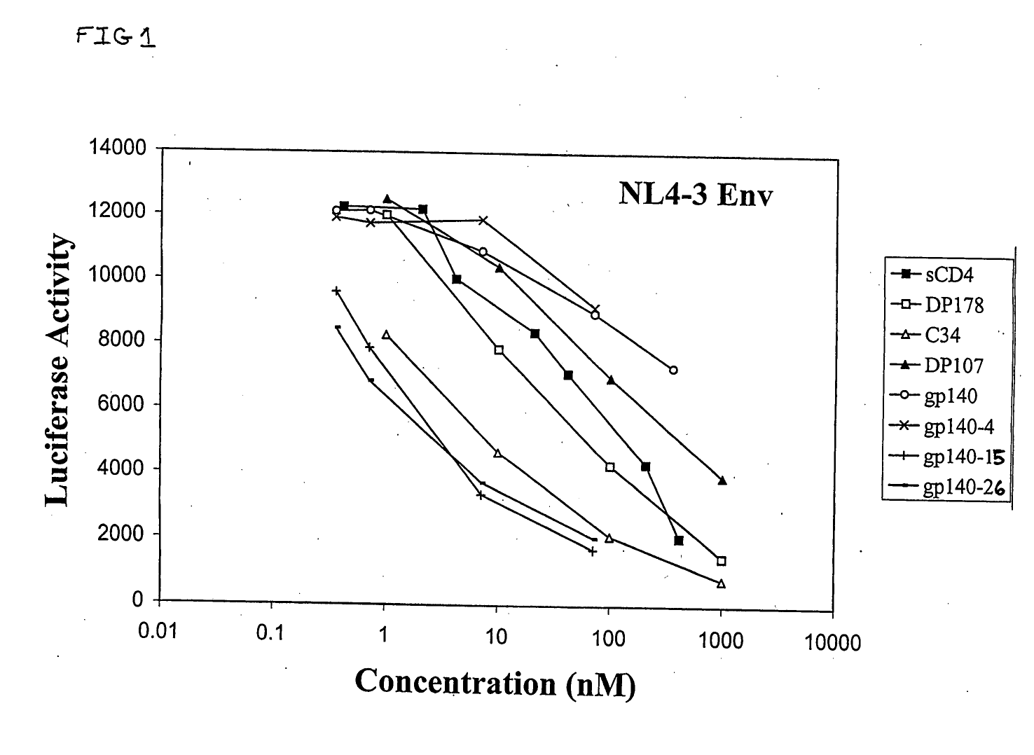

The results of these experimentss are shown in FIG. 1. FIG. 1 shows that gp140-26 is potent cell entry inhibitor.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Example 3

### Administration of the Fusion Protein Vaccine to a Human being

The 300 μg of gp120/41 fusion protein is vaccine prepared in an aluminum hydroxide adjuvant suspended in a sterile, isotonic buffered saline solution (as described in Cordonnier et al., Nature 340:571-574 (1989)). The preparation is then administered as an initial intramuscular injection, followed by identical boosters at 4, and 32 weeks, to an HIV serio-negative human of average height and weighing 75 Kg.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A human immunodeficiency virus antigenic composition comprising a human immunodeficiency virus envelope glycoprotein 160 having a gp120 subunit and a gp41 subunit wherein the carboxy-terminal end of gp120 is covalently linked through a peptide linker of at least 5 amino acids, to the amino-terminal end of gp41.

2. The antigenic composition of claim 1, wherein the human immunodeficiency virus envelope glycoprotein 160 is truncated at a position within 5 amino acids either side of amino acid 683 in SEQ ID NO:2.

3. The antigenic composition of claim 1, wherein the peptide linker is between 15 and 26 amino acids in length.

4. The antigenic composition of claim 1, wherein the peptide linker is selected from the group consisting of SEQ ID NO:10, SEQ ID NO:11, SEQ ID NO:12, SEQ ID NO:13, SEQ ID NO:14.

5. The antigenic composition of claim 1, wherein the human immunodeficiency virus envelope glycoprotein 160 has at least 70% amino acid sequence identity to sequence SEQ ID NO:2.

6. The antigenic composition of claim 1, wherein the human immunodeficiency virus envelope glycoprotein 160 is SEQ ID NO:7.

7. The antigenic composition of claim 2, wherein the human immunodeficiency virus envelope glycoprotein has at least 70% amino acid sequence identity to sequence SEQ ID NO:4.

8. The antigenic composition of claim 2, wherein the human immunodeficiency virus envelope glycoprotein is SEQ ID NO:8.

9. The antigenic composition of claim 1, wherein the gp120 subunit and the gp41 subunit are from different human immunodeficiency virus strains.

10. The antigenic composition of claim 1, wherein the gp120 subunit and the gp41 subunit are from the same human immunodeficiency virus strain.

11. A method of manufacturing a human immunodeficiency virus antigenic composition comprising a human immunodeficiency virus envelope glycoprotein 160 having a gp120 subunit and a gp41 subunit wherein the carboxy-terminal subunit of gp120 is covalently linked through a peptide linker of at least 5 amino acids to the amino terminal end of gp41, the method comprising:
(i) obtaining a nucleic acid encoding a gp120 and a gp 41.
(ii) introducing in frame between the gp120 and the gp41 coding segments a nucleic acid that encodes a peptide linker of between 6 and 29 amino acids, to yield a gene encoding a human immunodeficiency virus antigenic composition;
(iii) operably linking the gene to a expression cassette;
(iv) incorporating the expression cassette into a mammalian host cell;
(v) permitting the host to express the human immunodeficiency virus antigenic composition; and
(vi) isolating the composition from the host cell.

12. The method of claim 11, wherein the human immunodeficiency virus envelope glycoprotein 160 is truncated at a position within 5 amino acids either side of amino acid 683 in SEQ ID NO:2.

13. The method of claim 11, wherein the peptide linker is between 15 and 26 amino acids in length.

14. The method of claim 11, wherein the peptide linker is selected from the group consisting of SEQ ID NO:10, SEQ ID NO:11, SEQ ID NO:12, SEQ ID NO:13, SEQ ID NO:14.

15. The method of claim 11, wherein the human immunodeficiency vims envelope glycoprotein 160 has at least 70% amino acid sequence identity to sequence SEQ ID NO:2.

16. The antigenic composition of claim 1, wherein the human immunodeficiency virus envelope glycoprotein 160 is SEQ ID NO:7.

17. The method of claim 12, wherein the human immunodeficiency virus envelope glycoprotein has at least 70% amino acid sequence identity to sequence SEQ ID NO:4.

18. The method of claim 12, wherein the human immunodeficiency virus envelope glycoprotein is SEQ ID NO:8.

19. The method of claim 11, wherein the gp120 subunit and the gp41 subunit are from different human immunodeficiency virus strains.

20. The method of claim 11, wherein the gp120 subunit and the gp41 subunit are from the same human immunodeficiency virus strain.

21. A vaccine for protecting a human from human immunodeficiency virus infection comprising:
(i) an aliquot amount of a human immunodeficiency virus antigenic composition comprising a human immunodeficiency virus envelope glycoprotein 160 having a gp120 subunit and a gp41 subunit wherein the carboxy-terminal end of gp120 is covalently linked through a peptide linker of at least 5 amino acids to the amino-terminal end of gp41; and
(ii) a sterile pharmaceutically acceptable carrier.

22. The vaccine of claim 21, wherein the human immunodeficiency virus envelope glycoprotein 160 is truncated at a position within 5 amino acids either side of amino acid 683 in SEQ ID NO:2.

23. The vaccine of claim 21, wherein the peptide linker is between 15 and 26 amino acids in length.

24. The vaccine of claim 21, wherein the peptide linker is c selected from the group consisting of SEQ ID NO:10, SEQ ID NO:11, SEQ ID NO:12, SEQ ID NO:13, SEQ ID NO:14.

25. The vaccine of claim 21, wherein the human immunodeficiency virus envelope glycoprotein 160 has at least 70% amino acid sequence identity to sequence SEQ ID NO:2.

26. The vaccine of claim 21, wherein the human immunodeficiency virus envelope glycoprotein 160 is SEQ ID NO:7.

27. The vaccine of claim 22, wherein the human immunodeficiency virus envelope glycoprotein 160 has at least 70% amino acid sequence identity to sequence SEQ ID NO:4.

28. The vaccine of claim 22, wherein the human immunodeficiency virus envelope glycoprotein is SEQ ID NO:8.

29. The vaccine of claim 21, wherein the gp120 subunit and the gp41 subunit are from different human immunodeficiency virus strains.

30. The vaccine of claim 21, wherein the gp120 subunit and the gp41 subunit are from the same human immunodeficiency virus strain.

31. The vaccine of claim 21, wherein the aliquot amount of human immunodeficiency virus antigenic composition is between 0.5 and 1 milligrams antigenic composition per milliliter of sterile pharmaceutically acceptable carrier.

32. The vaccine of claim 21, wherein the aliquot amount of human immunodeficiency virus antigenic composition is in a lyophilized state.

33. A method of protecting a human from human immunodeficiency virus infection comprising:
administering to a human an amount of a human immunodeficiency virus antigenic composition comprising a human immunodeficiency virus envelope glycoprotein 160 having a gp120 subunit and a gp41 subunit, wherein the carboxy-terminal end of gp120 is covalently linked through a peptide linker of at least 5 amino acids to the amino-terminal end of gp41, wherein the amount administered is effective to immunize the human against human immunodeficiency virus infection.

34. The method of claim 33, wherein the human immunodeficiency virus envelope glycoprotein 160 is truncated at a position within 5 amino acids either side of amino acid 683 in SEQ ID NO:2.

35. The method of claim 33, wherein the peptide linker is between 15 and 26 amino acids in length.

36. The method of claim 33, wherein the peptide linker is selected from the group consisting of SEQ ID NO:10, SEQ ID NO:11, SEQ ID NO:12, SEQ ID NO:13, SEQ ID NO:14.

37. The method of claim 33, wherein the human immunodeficiency virus envelope glycoprotein 160 has at least 70% amino acid sequence identity to sequence SEQ ID NO:2.

38. The method of claim 33, wherein the human immunodeficiency virus envelope glycoprotein 160 is SEQ ID NO:7.

39. The method of claim 34, wherein the human immunodeficiency virus envelope glycoprotein has at least 70% amino acid sequence identity to sequence SEQ ID NO:4.

40. The method of claim 34, wherein the human immunodeficiency virus envelope glycoprotein is SEQ ID NO:8.

41. The method of claim 33, wherein the gp120 subunit and the gp41 subunit are from different human immunodeficiency virus strains.

42. The method of claim 33, wherein the gp120 subunit and the gp41 subunit are from the same human immunodeficiency virus strain.

43. The method of claim 33, wherein the amount administered effective to immunize the human against human immunodeficiency virus infection is between 1 μg/kg and 20 μg/kg per dose per innoculation.

44. The method of claim 33, wherein the human immunodeficiency virus antigenic composition further comprises one or more glycoprotein 160 ligands chosen from the group consisting of CD4, CCR5 and CXCR4.

45. The method of claim 44, wherein the molar ration of glycoprotein 160 to ligand is between 3:1 and 1:3 for each ligand species of the composition.

46. An nucleic acid comprising a coding sequence for a human immunodeficiency virus envelope glycoprotein 160 having a gp120 subunit and a gp41 subunit wherein the carboxy-terminal end of gp120 is covalently linked through a peptide linker of at least 5 amino acids to the amino-terminal end of gp41.

47. A live recombinant vaccine comprising an nucleic acid comprising a coding sequence for a human immunodeficiency virus envelope glycoprotein 160 having a gp120 subunit and a gp41 subunit wherein the carboxy-terminal end of gp120 is covalently linked through a peptide linker of at least 5 amino acids to the amino-terminal end of gp41.

48. The nucleic acid of claim 46, further comprising regulatory sequences for the expression of DNA in eukaryotic cells operably linked to the human immunodeficiency virus envelope glycoprotein 160 sequence.

49. The live recombinant vaccine of claim 47, further comprising regulatory sequences for the expression of DNA in eukaryotic cells operably linked to the human immunodeficiency virus envelope glycoprotein 160 sequence.

50. The antigenic composition of claim 1, wherein the human immunodeficiency virus envelope glycoprotein 160 comprises the extracellular subunits of envelope glycoprotein 160.

51. The vaccine of claim 21, wherein the human immunodeficiency virus envelope glycoprotein 160 comprises the extracellular subunits of envelope glycoprotein 160.

52. The method of claim 33, wherein the human immunodeficiency virus envelope glycoprotein 160 comprises the extracellular subunits of envelope glycoprotein 160.

53. The nucleic acid of claim 46, wherein the human immunodeficiency virus envelope glycoprotein 160 comprises the extracellular subunits of envelope glycoprotein 160.

54. The live recombinant vaccine of claim 47, wherein the human immunodeficiency virus envelope glycoprotein 160 comprises the extracellular subunits of envelope glycoprotein 160.

55. The human immunodeficiency virus antigenic composition of claim 1, wherein the peptide linker is of 6 to 29 amino acids.

56. The method of claim 11, wherein the peptide linker is of 6 to 29 amino acids.

57. The method of claim 33, wherein the peptide linker is of 6 to 29 amino acids.

58. The nucleic acid of claim 46, wherein the peptide linker is of 6 to 29 amino acids.

59. The live recombinant vaccine of claim 47, wherein the peptide linker is of 6 to 29 amino acids.

60. The nucleic acid of claim 46, wherein the human immunodeficiency virus envelope glycoprotein 160 sequence is SEQ ID NO:7 or SEQ ID NO:8.

61. The live recombinant vaccine of claim 47, wherein the human immunodeficiency virus envelope glycoprotein 160 sequence is SEQ ID NO:7 or SEQ ID NO:8.

62. The nucleic acid of claim 46, further comprising a nucleic acid encoding one or more glycoprotein 160 ligands chosen from the group consisting of CD4, CCR5 and CXCR4.

63. The live recombinant vaccine of claim 47, further comprising one or more glycoprotein 160 ligands chosen from the group consisting of CD4, CCR5 and CXCR4.

64. The vaccine of claim 21, further comprising (iii) one or more glycoprotein 160 ligands chosen from the group consisting of CD4, CCR5 and CXCR4.

65. The method of claim 33, whenrein the antigenic composition further comprises one or more glycoprotein 160 ligands chosen from the group consisting of CD4, CCR5 and CXCR4.

66. The method of claim 11, wherein the gp41 subunit is an extracellular subunit(s) of gp41.

67. The nucleic acid of claim 46, wherein the peptide linker is between 15 and 26 amino acids in length.

68. The nucleic acid of claim 46, wherein the peptide linker is selected from the group consisting of SEQ ID NO:10, SEQ ID NO:11, SEQ ID NO:12, SEQ ID NO:13, SEQ ID NO:14.

69. The nucleic acid of claim 46, wherein the human immunodeficiency virus envelope glycoprotein 160 has at least 70% amino acid sequence identity to sequence SEQ ID NO:2.

70. The nucleic acid of claim 46, wherein the human immunodeficiency virus envelope glycoprotein 160 is SEQ ID NO:7.

71. The nucleic acid of claim 46, wherein the human immunodeficiency virus envelope glycoprotein has at least 70% amino acid sequence identity to sequence SEQ ID NO:4.

72. The nucleic acid of claim 46, wherein the human immunodeficiency virus envelope glycoprotein is SEQ ID NO:8.

73. The nucleic acid of claim 46, wherein the gp120 subunit and the gp41 subunit are from different human immunodeficiency virus strains.

74. The nucleic acid of claim 46, wherein the gp120 subunit and the gp41 subunit are from the same human immunodeficiency virus strain.

75. The live recombinant vaccine of claim 47, wherein the peptide linker is between 15 and 26 amino acids in length.

76. The live recombinant vaccine of claim 47, wherein the gp120 subunit and the gp41 subunit are from the same human immunodeficiency virus strain.

77. The live recombinant vaccine of claim 47, wherein the peptide linker is selected from the group consisting of SEQ ID NO:10, SEQ ID NO:11, SEQ ID NO:12, SEQ ID NO:13, SEQ ID NO:14.

78. The live recombinant vaccine of claim 47, wherein the human immunodeficiency virus envelope glycoprotein 160 has at least 70% amino acid sequence identity to sequence SEQ ID NO:2.

79. The live recombinant vaccine of claim 47, wherein the human immunodeficiency virus envelope glycoprotein 160 is SEQ ID NO:7.

80. The live recombinant vaccine of claim 47, wherein the human immunodeficiency virus envelope glycoprotein has at least 70% amino acid sequence identity to sequence SEQ ID NO:4.

81. The live recombinant vaccine of claim 47, wherein the human immunodeficiency virus envelope glycoprotein is SEQ ID NO:8.

82. The live recombinant vaccine of claim 47, wherein the gp120 subunit and the gp41 subunit are from different human immunodeficiency virus strains.

83. The nucleic acid of claim 62, wherein the molar ration of glycoprotein 160 to ligand is between 3:1 and 1:3 for each ligand species of the composition.

84. The live recombinant vaccine of claim 63, wherein the molar ration of glycoprotein 160 to ligand is between 3:1 and 1:3 for each ligand species of the composition.


SEQUENCE LISTING (33 sequences)

SEQ ID NO: 1 - DNA, 2562 bp, Human immunodeficiency virus type 1
  full length human immunodeficiency virus-1 (HIV-1) envelope glycoprotein (Env, gp160) from primary R5X4 isolate 89.6
atgagagtgaaggagatcaggaagaattggcagcacttgagagggggcatcttgctccttgggatgttgatgatctgtagtgctgcaaaagaaaaaacgtgggtcacaatctattatggggtacctgtgtggagagaagcaaccaccactctattttgtgcatcagatgctaaagcctatgatacagaggtacataatgtttgggccacacatgcctgtgtacccacagaccccaacccacaagaagtagtattgggaaatgtgacagaaaattttaacatgtggaaaaataacatggtagatcagatgcatgaggatataatcagtttatgggatgaaagcctaaagccatgtgtaaaattaaccccactctgtgttactttaaattgcactaatttgaatatcactaagaatactactaatcccactagtagcagctggggaatgatggagaaaggagaaataaaaaattgctctttctatatcaccacaagcataagaaataaggtaaagaaagaatatgcactttttaatagacttgatgtagtaccaatagaaaatactaataatactaagtataggttaataagttgtaacacctcagtcattacacaggcctgtccaaaggtatcctttcagccaattcccatacattattgtgtcccggctgggtttgcgatgctaaagtgtaacaataagacattcaatggatcaggaccatgcacaaatgtcagcacagtacaatgtacacatggaattaggccagtggtgtcaactcaactgctgttaaatggcagtctagcagaagaagacatagtaattagatctgaaaatttcacagacaatgctaaaaccataatagtacagctaaatgaatctgtagtaattaattgtacaagacccaacaacaatacaagaagaaggttatctataggaccagggagagcattttatgcaagaagaaacataataggagatataagacaagcacattgtaacattagtagagcaaaatggaataacactttacaacagatagttataaaattaagagaaaaatttaggaataaaacaatagcctttaatcaatcctcaggaggggacccagaaattgtaatgcacagttttaattgtggaggggaatttttctactgtaatacagcacaactgtttaatagtacttggaatgttactggagggacaaatggcactgaaggaaatgacataatcacactccaatgcagaataaaacaaattataaatatgtggcagaaagtaggaaaagcaatgtatgcccctcccatcacaggacaaattagatgttcatcaaatattacagggctgctactaacaagagatggaggtaatagtactgagactgagactgagatcttcagacctggaggaggagatatgagggacaattggagaagtgaattatataaatataaagtagtaagaattgaaccaataggagtagcacccaccagggcaaagagaagaacagtgcaaagagaaaaaagagcagtgggaataggagctgtgttccttgggttcttgggagcagcaggaagcactatgggcgcagcgtcagtgacgctgacggtacaggccaggctattattgtctggtatagtgcagcagcagaacaatctgctgagggctattgaggcgcaacagcatatgttgcaactcacagtctggggcatcaagcagctccaggcaagagtcctggctctggaaagatacctaagggatcaacagctcatgggaatttggggttgctctggaaaactcatttgcaccacttctgtgccttggaatgttagttggagtaataaatctgtggatgatatttggaataacatgacctggatggagtgggaaagagaaattgacaattacacagactatatatatgacttacttgaaaaatcgcaaacccaacaagaaaagaatgaaaaagaattattggaattggataaatgggcaagtttgtggaattggtttgacataacaaactggctgtggtatataagattattcataatgatagtaggaggcttgataggtttaagaatagtttttgctgtactttctatagtaaatagagttaggcagggatattcaccattatcgtttcagaccctcctcccagcctcgaggggacccgacaggcccgaaggaacagaagaagaaggtggagagagagacagagacagatccggtccattagtgaacggattcttggcacttttctgggtcgatttgaggaacctgtgcctcttcctctaccacctcttgagaaacttactcttgattgtaacgaggattgtggaacttctgggacgcagggggtgggaagccctcaaatattggtggaatctcctgcaatattggagccaggaactaaagaatagtgctgttagcttgctcaacgccacagccatagcagtagctgaggggacagatagggttataaaaatagtacaaagagcttgtagagctattcgcaacatacctacaagaatcagacagggcttggaaagggctttgctataa

SEQ ID NO: 2 - protein, 853 aa, Human immunodeficiency virus type 1
  full length human immunodeficiency virus-1 (HIV-1) envelope glycoprotein (Env, gp160) from primary R5X4 isolate 89.6
MRVKEIRKNWQHLRGGILLLGMLMICSAAKEKTWVTIYYGVPVWREATTTLFCASDAKAYDTEVHNVWATHACVPTDPNPQEVVLGNVTENFNMWKNNMVDQMHEDIISLWDESLKPCVKLTPLCVTLNCTNLNITKNTTNPTSSSWGMMEKGEIKNCSFYITTSIRNKVKKEYALFNRLDVVPIENTNNTKYRLISCNTSVITQACPKVSFQPIPIHYCVPAGFAMLKCNNKTFNGSGPCTNVSTVQCTHGIRPVVSTQLLLNGSLAEEDIVIRSENFTDNAKTIIVQLNESVVINCTRPNNNTRRRLSIGPGRAFYARRNIIGDIRQAHCNISRAKWNNTLQQIVIKLREKFRNKTIAFNQSSGGDPEIVMHSFNCGGEFFYCNTAQLFNSTWNVTGGTNGTEGNDIITLQCRIKQIINMWQKVGKAMYAPPITGQIRCSSNITGLLLTRDGGNSTETETEIFRPGGGDMRDNWRSELYKYKVVRIEPIGVAPTRAKRRTVQREKRAVGIGAVFLGFLGAAGSTMGAASVTLTVQARLLLSGIVQQQNNLLRAIEAQQHMLQLTVWGIKQLQARVLALERYLRDQQLMGIWGCSGKLICTTSVPWNVSWSNKSVDDIWNNMTWMEWEREIDNYTDYIYDLLEKSQTQQEKNEKELLELDKWASLWNWFDITNWLWYIRLFIMIVGGLIGLRIVFAVLSIVNRVRQGYSPLSFQTLLPASRGPDRPEGTEEEGGERDRDRSGPLVNGFLALFWVDLRNLCLFLYHLLRNLLLIVTRIVELLGRRGWEALKYWWNLLQYWSQELKNSAVSLLNATAIAVAEGTDRVIKIVQRACRAIRNIPTRIRQGLERALL

SEQ ID NO: 3 - DNA, 2051 bp, Human immunodeficiency virus type 1
  human immunodeficiency virus-1 (HIV-1) envelope glycoprotein gp140 truncated version of gp 160 from primary R5X4 isolate 89.6
atgagagtgaaggagatcaggaagaattggcagcacttgagagggggcatcttgctccttgggatgttgatgatctgtagtgctgcaaaagaaaaaacgtgggtcacaatctattatggggtacctgtgtggagagaagcaaccaccactctattttgtgcatcagatgctaaagcctatgatacagaggtacataatgtttgggccacacatgcctgtgtacccacagaccccaacccacaagaagtagtattgggaaatgtgacagaaaattttaacatgtggaaaaataacatggtagatcagatgcatgaggatataatcagtttatgggatgaaagcctaaagccatgtgtaaaattaaccccactctgtgttactttaaattgcactaatttgaatatcactaagaatactactaatcccactagtagcagctggggaatgatggagaaaggagaaataaaaaattgctctttctatatcaccacaagcataagaaataaggtaaagaaagaatatgcactttttaatagacttgatgtagtaccaatagaaaatactaataatactaagtataggttaataagttgtaacacctcagtcattacacaggcctgtccaaaggtatcctttcagccaattcccatacattattgtgtcccggctgggtttgcgatgctaaagtgtaacaataagacattcaatggatcaggaccatgcacaaatgtcagcacagtacaatgtacacatggaattaggccagtggtgtcaactcaactgctgttaaatggcagtctagcagaagaagacatagtaattagatctgaaaatttcacagacaatgctaaaaccataatagtacagctaaatgaatctgtagtaattaattgtacaagacccaacaacaatacaagaagaaggttatctataggaccagggagagcattttatgcaagaagaaacataataggagatataagacaagcacattgtaacattagtagagcaaaatggaataacactttacaacagatagttataaaattaagagaaaaatttaggaataaaacaatagcctttaatcaatcctcaggaggggacccagaaattgtaatgcacagttttaattgtggaggggaatttttctactgtaatacagcacaactgtttaatagtacttggaatgttactggagggacaaatggcactgaaggaaatgacataatcacactccaatgcagaataaaacaaattataaatatgtggcagaaagtaggaaaagcaatgtatgcccctcccatcacaggacaaattagatgttcatcaaatattacagggctgctactaacaagagatggaggtaatagtactgagactgagactgagatcttcagacctggaggaggagatatgagggacaattggagaagtgaattatataaatataaagtagtaagaattgaaccaataggagtagcacccaccagggcaaagagaagaacagtgcaaagagaaaaaagagcagtgggaataggagctgtgttccttgggttcttgggagcagcaggaagcactatgggcgcagcgtcagtgacgctgacggtacaggccaggctattattgtctggtatagtgcagcagcagaacaatctgctgagggctattgaggcgcaacagcatatgttgcaactcacagtctggggcatcaagcagctccaggcaagagtcctggctctggaaagatacctaagggatcaacagctcatgggaatttggggttgctctggaaaactcatttgcaccacttctgtgccttggaatgttagttggagtaataaatctgtggatgatatttggaataacatgacctggatggagtgggaaagagaaattgacaattacacagactatatatatgacttacttgaaaaatcgcaaacccaacaagaaaagaatgaaaaagaattattggaattggataaatgggcaagtttgtggaattggtttgacataacaaactggctgtggtatataagattattcataat

SEQ ID NO: 4 - protein, 667 aa, Human immunodeficiency virus type 1
  human immunodeficiency virus-1 (HIV-1) envelope glycoprotein gp140 truncated version of gp 160 from primary R5X4 isolate 89.6
MRVKEIRKNWQHLRGGILLLGMLMICSAAKEKTWVTIYYGVPVWREATTTLFCASDAKAYDTEVHNVWATHACVPTDPNPQEVVLGNVTENFNMWKNNMVDQMHEDIISLWDESLKPCVKLTPLCVTLNCTNLNITKNTTNPTSSSWGMMEKGEIKNCSFYITTSIRNKVKKEYALFNRLDVVPIENTNNTKYRLISCNTSVITQACPKVSFQPIPIHYCVPAGFAMLKCNNKTFNGSGPCTNVSTVQCTHGIRPVVSTQLLLNGSLAEEDIVIRSENFTDNAKTIIVQLNESVVINCTRPNNNTRRRLSIGPGRAFYARRNIIGDIRQAHCNISRAKWNNTLQQIVIKLREKFRNKTIAFNQSSGGDPEIVMHSFNCGGEFFYCNTAQLFNSTWNVTGGTNGTEGNDIITLQCRIKQIINMWQKVGKAMYAPPITGQIRCSSNITGLLLTRDGGNSTETETEIFRPGGGDMRDNWRSELYKYKVVRIEPIGVAPTRAKRRTVQREKRAVGIGAVFLGFLGAAGSTMGAASVTLTVQARLLLSGIVQQQNNLLRAIEAQQHMLQLTVWGIKQLQARVLALERYLRDQQLMGIWGCSGKLICTTSVPWNVSWSNKSVDDIWNNMTWMEWEREIDNYTDYIYDLLEKSQTQQEKNEKELLELDKWASLW

SEQ ID NO: 5 - DNA, 27 bp, Artificial Sequence
  Description of Artificial Sequencecleavage site and start of gp41 in gp160 (env 89.6)
caaagagaaaaaagagcagtgggaata

SEQ ID NO: 6 - protein, 8 aa, Artificial Sequence
  Description of Artificial Sequencecleavage site and start of gp41 in gp160 (env 89.6)
REKRAVGI

SEQ ID NO: 7 - protein, 667 aa, Artificial Sequence
  Description of Artificial Sequencegp140-26 (soluble, secreted protein)
KEKTWVTIYYGVPVWREATTTLFCASDAKAYDTEVHNGWATHACVATDPNPQEVVLGNVTENFNMWKNNMVDQMHEDIISLLDESLKPCVKLTPLCVTLNCTNLNITKNTTNPTSSSLGMMEKGEIKNCSFYITTSIRNKVKKEYALFNRLDVVPIENTNNTKYRLISCNTSVITQACPKVFFQPIAIHYCVPAGFAMLKCNNKTFNGSGPCTNVSTVPCTHGIRPVVSTQLLLNGSLAEEDIVIRSGNFTDNAKTIIVQLNESVVINCTRPNNNTRRRLSIGPGRAFYARRNIIGDIRQAHCNISRAKLNNTLQQIVIKLREKFRNKTIAFNQSSGGDPEIVMHSFNCGGEFFYCNTAQLFNSTLNVTGGTNGTEENDIITLQCRIKQIINMWQKVGKAMYAPPITGQIICSSNITGLLLTRDGGNSTETETEIFRPGGGDMRDNWRSELYKYKVVRIEPIGVAPTRAKRRTCQGGIDGILQISGSGSGGSGQGSSSGGAGGKGAVGIGAVFLGFLGAAGSTMGARSVTLTVQARLLLSGIVQQQNNLLRAIEAQQHMLQLTVWGIKQLQARVLALERYLRDQQLMGIWGCSGKLICTTSVPWNVSWSNKSVDDIWNNMTWMELEREIDNYTDYIYDLLEKSQTQQEKNEKELLELDKWASLWKLV

SEQ ID NO: 8 - protein, 656 aa, Artificial Sequence
  Description of Artificial Sequencegp140-15 (soluble, secreted protein)
KEKTWVTIYYGVPVWREATTTLFCASDAKAYDTEVHNGWATHACVATDPNPQEVVLGNVTENFNMWKNNMVDQMHEDIISLLDESLKPCVKLTPLCVTLNCTNLNITKNTTNPTSSSLGMMEKGEIKNCSFYITTSIRNKVKKEYALFNRLDVVPIENTNNTKYRLISCNTSVITQACPKVFFQPIAIHYCVPAGFAMLKCNNKTFNGSGPCTNVSTVPCTHGIRPVVSTQLLLNGSLAEEDIVIRSGNFTDNAKTIIVQLNESVVINCTRPNNNTRRRLSIGPGRAFYARRNIIGDIRQAHCNISRAKLNNTLQQIVIKLREKFRNKTIAFNQSSGGDPEIVMHSFNCGGEFFYCNTAQLFNSTLNVTGGTNGTEENDIITLQCRIKQIINMWQKVGKAMYAPPITGQIICSSNITGLLLTRDGGNSTETETEIFRPGGGDMRDNWRSELYKYKVVRIEPIGVAPTRAKRRTCQGGIDGILQISSSGGAGGKGAVGIGAVFLGFLGAAGSTMGARSVTLTVQARLLLSGIVQQQNNLLRAIEAQQHMLQLTVWGIKQLQARVLALERYLRDQQLMGIWGCSGKLICTTSVPWNVSWSNKSVDDIWNNMTWMELEREIDNYTDYIYDLLEKSQTQQEKNEKELLELDKWASLWKLV

SEQ ID NO: 9 - protein, 4 aa, Artificial Sequence
  Description of Artificial Sequencepeptide linker
GILI

SEQ ID NO: 10 - protein, 15 aa, Artificial Sequence
  Description of Artificial Sequencepreferred peptide linker
GGIDGILQISSSGGA

SEQ ID NO: 11 - protein, 26 aa, Artificial Sequence
  Description of Artificial Sequencepreferred peptide linker
GGIDGILQISGSGSGGSGQGSSSGGA

SEQ ID NO: 12 - protein, 15 aa, Artificial Sequence
  Description of Artificial Sequencepreferred peptide linker
GGSGGGGSGGGGSGG

SEQ ID NO: 13 - protein, 20 aa, Artificial Sequence
  Description of Artificial Sequencepreferred peptide linker
GGSGGGGSGGGGSGGGGSGG

SEQ ID NO: 14 - protein, 25 aa, Artificial Sequence
  Description of Artificial Sequencepreferred peptide linker
GGSGGGGSGGGGSGGGGSGGGGSGG

SEQ ID NO: 15 - protein, 24 aa, Artificial Sequence
  Description of Artificial Sequenceexemplary linker
GSGSGSGSGSGSGSGSGSGSGSGS

SEQ ID NO: 16 - protein, 48 aa, Artificial Sequence
  Description of Artificial Sequenceexemplary linker
GGGSGGGSGGGSGGGSGGGSGGGSGGGSGGGSGGGSGGGSGGGSGGGS

SEQ ID NO: 17 - protein, 60 aa, Artificial Sequence
  Description of Artificial Sequenceexemplary linker
GGGGSGGGGSGGGGSGGGGSGGGGSGGGGSGGGGSGGGGSGGGGSGGGGSGGGGSGGGGS

SEQ ID NO: 18 - protein, 72 aa, Artificial Sequence
  Description of Artificial Sequenceexemplary linker
GGGGGSGGGGGSGGGGGSGGGGGSGGGGGSGGGGGSGGGGGSGGGGGSGGGGGSGGGGGSGGGGGSGGGGGS

SEQ ID NO: 19 - protein, 12 aa, Artificial Sequence
  Description of Artificial Sequenceexemplary linker
GGGSGGGSGGGS

SEQ ID NO: 20 - protein, 20 aa, Artificial Sequence
  Description of Artificial Sequenceexemplary linker
GGGGSGGGGSGGGGSGGGGS

SEQ ID NO: 21 - protein, 30 aa, Artificial Sequence
  Description of Artificial Sequenceexemplary linker
GGGGGSGGGGGSGGGGGSGGGGGSGGGGGS

SEQ ID NO: 22 - protein, 42 aa, Artificial Sequence
  Description of Artificial Sequenceexemplary linker
GGGGGGSGGGGGGSGGGGGGSGGGGGGSGGGGGGSGGGGGGS

SEQ ID NO: 23 - protein, 56 aa, Artificial Sequence
  Description of Artificial Sequenceexemplary linker
GGGGGGGSGGGGGGGSGGGGGGGSGGGGGGGSGGGGGGGSGGGGGGGSGGGGGGGS

SEQ ID NO: 24 - protein, 72 aa, Artificial Sequence
  Description of Artificial Sequenceexemplary linker
GGGGGGGGSGGGGGGGGSGGGGGGGGSGGGGGGGGSGGGGGGGGSGGGGGGGGSGGGGGGGGSGGGGGGGGS

SEQ ID NO: 25 - protein, 90 aa, Artificial Sequence
  Description of Artificial Sequenceexemplary linker
GGGGGGGGGSGGGGGGGGGSGGGGGGGGGSGGGGGGGGGSGGGGGGGGGSGGGGGGGGGSGGGGGGGGGSGGGGGGGGGSGGGGGGGGGS

SEQ ID NO: 26 - protein, 110 aa, Artificial Sequence
  Description of Artificial Sequenceexemplary linker
GGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGS

SEQ ID NO: 27 - protein, 132 aa, Artificial Sequence
  Description of Artificial Sequenceexemplary linker
GGGGGGGGGGGSGGGGGGGGGGGSGGGGGGGGGGGSGGGGGGGGGGGSGGGGGGGGGGGSGGGGGGGGGGGSGGGGGGGGGGGSGGGGGGGGGGGSGGGGGGGGGGGSGGGGGGGGGGGSGGGGGGGGGGGS

SEQ ID NO: 28 - protein, 156 aa, Artificial Sequence
  Description of Artificial Sequenceexemplary linker
GGGGGGGGGGGGSGGGGGGGGGGGGSGGGGGGGGGGGGSGGGGGGGGGGGGSGGGGGGGGGGGGSGGGGGGGGGGGGSGGGGGGGGGGGGSGGGGGGGGGGGGSGGGGGGGGGGGGSGGGGGGGGGGGGSGGGGGGGGGGGGSGGGGGGGGGGGGS

SEQ ID NO: 29 - protein, 36 aa, Artificial Sequence
  Description of Artificial Sequenceexemplary linker
AGSAGSAGSAGSAGSAGSAGSAGSAGSAGSAGSAGS

SEQ ID NO: 30 - protein, 4 aa, Artificial Sequence
  Description of Artificial Sequencepost translational cleavage site
REKR

SEQ ID NO: 31 - protein, 4 aa, Artificial Sequence
  Description of Artificial Sequencemutated post translational cleavage site
REID

SEQ ID NO: 32 - protein, 4 aa, Artificial Sequence
  Description of Artificial Sequenceshort fragment following mutated cleavage site created by introduction of restriction sites
EFIS

SEQ ID NO: 33 - protein, 5 aa, Artificial Sequence
  Description of Artificial Sequencepolypeptide linker end
GGSGG